(19) United States
(12) Patent Application Publication
(10) Pub. No.: US 2017/0202802 A1
(43) Pub. Date: Jul. 20, 2017

(54) COMPOSITIONS, KITS AND METHODS FOR NUTRITION SUPPLEMENTATION

(71) Applicant: Exeltis USA, Inc., Florham Park, NJ (US)

(72) Inventors: Eduardo Fernandez, Hoboken, NJ (US); Charles Balzer, Chatham, NJ (US)

(21) Appl. No.: 15/002,266

(22) Filed: Jan. 20, 2016

(51) Int. Cl. A61K 31/225; A61K 33/34; A61K 31/07; A61K 31/375; A61K 31/355; A61K 31/51; A61K 31/525; A61K 31/455; A61K 31/675; A61K 31/506; A61K 31/714; A61K 31/715; A61K 33/18; A61K 33/08; A61K 33/30; A61K 31/202; A61K 36/02; A61K 9/48

(52) U.S. Cl. CPC A61K 31/225; A61K 9/4891; A61K 33/34; A61K 31/07; A61K 31/375; A61K 31/355; A61K 31/51; A61K 31/525; A61K 31/455; A61K 31/675; A61K 31/506; A61K 31/714; A61K 31/715; A61K 33/18; A61K 33/08; A61K 33/30; A61K 31/202; A61K 36/02; A23L 1/302; A23L 1/303; A23L 1/304; A23V 2002/00

(57) ABSTRACT

Kits, compositions and methods are provided which comprise a nutritional composition and an adjuvant composition. The nutritional composition comprises vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids and one or more pharmaceutically-acceptable carriers. The adjuvant composition is formulated to mitigate at least one undesired side effect associated with the administering of the first composition to the patient. The first composition may comprise at least about 90 mg of iron. The iron may be provided in the form of a polysaccharide iron complex. The adjuvant composition may comprise fiber or a laxative to mitigate the undesired side effects of the relatively high iron content of the nutritional composition.

### FIELD OF THE INVENTION

The present invention relates to various vitamin, nutrient and mineral compositions and kits for nutritional supplementation and methods of administration of compositions and kits for nutritional supplementation in, for example, subjects in physiologically stressful states, such as occur during pregnancy, lactation, or in need thereof.

### BACKGROUND

Nutrition plays a critical role in maintaining good health. Proper nutrition prevents dietary deficiencies, and also protects against the development of disease. When the body faces physiological stress, proper nutrition plays an increasingly important role. For example, pregnancy and lactation are among the most nutritionally volatile and physiologically stressful periods and processes in the lifetimes of women. Vitamin and mineral needs are almost universally increased during these natural processes. Increased vitamin and mineral needs during these times are almost always due to elevated metabolic demand, increased plasma volume, increased levels of blood cells, decreased concentrations of nutrients, and decreased concentrations of nutrient-binding proteins.

When increased nutrient needs occur during pregnancy, lactation, or any other physiologically stressful state, nutritional supplementation serves a vital role in maintaining good health. Nutritional supplementation is especially pertinent to women contemplating conceiving a child because optimizing specific nutrients before, during, and after the physiological processes of pregnancy or lactation can have profound, positive, and comprehensive impacts upon the overall wellness of the developing and newborn child as well as on the safety and health of the mother.

### BRIEF SUMMARY

The present invention provides compositions and methods of administering compositions for both prophylactic and therapeutic nutritional supplementation. Specifically, for example, the present invention relates to novel compositions of vitamins, minerals, and omega-3 fatty acids that can be used to supplement the nutritional deficiencies observed in patients throughout physiologically stressful states, which, in certain embodiments of the present invention, include prenatal, pregnant and breast-feeding women.

In one embodiment, a kit is provided. The kit may comprise a first outer coating encapsulating a first filler composition comprising vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids, and one or more pharmaceutically-acceptable carriers. The kit may further comprise a second outer coating encapsulating a second filler composition comprising a laxative and one or more pharmaceutically-acceptable carriers.

In a separate aspect, the first filling composition may comprise at least about 1100 IU vitamin A, at least about 60 mg vitamin C, at least about 1000 IU vitamin D, at least about 20 IU vitamin E, at least about 1.6 mg vitamin B1, at least about 1.8 mg B2, at least about 15 mg B3, at least about 1 mg vitamin B9, at least about 25 μg vitamin B12, at least about 90 mg iron, at least about 150 μg iodine, at least about 20 mg magnesium, at least about 25 mg zinc, at least about 2 mg copper and at least about 400 mg of the source of omega-3 fatty acids.

In another separate aspect, the vitamin A may be in the form of beta carotene, the vitamin C may be in the form of ascorbic acid, the vitamin D may be in form of cholecalciferol, the vitamin E may be in the form of dl-alpha tocopheryl acetate, the vitamin B1 may be in the form of thiamin, the vitamin B2 may be in the form of riboflavin, the vitamin B3 may be in the form of niacinamide, the vitamin B6 may be in the form of pyridoxine hydrochloride, the vitamin B9 may be provided as a combination of folic acid and L-methylfolate calcium, the vitamin B12 may be in the form of cyanocobalamin, the iron may be in the form of polysaccharide iron complex, the iodine may be in the form of potassium iodide, the magnesium may be in the form of magnesium oxide, the zinc may be in the form of zinc oxide, the copper may be in the form of copper oxide, the source of omega-3 fatty acids may be an algal oil comprising Crypthecodinium cohnii.

In another separate aspect, the vitamin A may be in the form selected from one or more of the group consisting of retinol acetate, retinol, retinol palmitate, retinoic acid, retinal, beta-cryptoxanthin, alpha-carotene, beta-carotene, gamma-carotene, and provitamin A carotenoids.

In another separate aspect, the vitamin C may be in the form selected from one or more of the group consisting of ascorbic acid, asorbates, calcium ascorbate, sodium ascorbate, dehydroascorbic acid and salts, ascorbyl palmitate, ascorbyl phosphates and salts, ascorbyl sulfates and salts, acylated ascorbic acid derivatives, 6-bromo-6-deoxy-L-ascorbic acid, and ascorbate salts.

In another separate aspect, the vitamin D may be in the form selected from one or more of the group consisting of vitamin D3, vitamin D2, previtamin D2, ergosterol, calcitriol, 7-dehydrocholesterol, Vitamin D1, vitamin D4, vitamin, 7-dehydrositosterol, Lumisterol, 25-hydroxyvitamin D, all steroids that exhibit the biological activity of calciol, 25-fluorocalciol, (3S)-3-amino-3-deoxycalciol, 11α-acetoxycalciol, calcidiol, ercalcitriol, calcitetrol, tacalciol, (5E)-isocalciol, Dihydroercalciol, (1S)-Hydroxycalciol, (24R)-Hydroxycalcidiol, Ercalcidiol, Ercalcitriol, Ertacalciol, (5E)-(10S)-10,19-Dihydroercalciol, (6Z)-Tacalciol, and (22E)-(24R)-Ethyl-22,23-didehydrocalciol.

In another separate aspect, the vitamin E may be in the form selected from one or more of the group consisting of alpha, beta, gamma, and delta tocopherols in its natural or synthetic (dl) forms; alpha, beta, gamma, and delta tocotrienols in its natural or synthetic (dl) forms, dl-alpha tocopheryl derivatives such as dl-alpha tocopheryl esters, dl-alpha-tocopheryl acetate or succinate and d-alpha-tocopheryl acetate or dl-alpha tocopheryl phosphates.

In another separate aspect, the vitamin B1 may be in the form selected from one or more of the group consisting of thiamine, thiamine mononitrate, thiamine monophosphate, thiamine diphosphate, thiamine triphosphate, acetiamine, allithiamine, prosultiamine and S-acyl derivatives of thiamine such as benfotiamine, fursultiamine and salts and esters thereof.

In another separate aspect, the vitamin B2 may be in the form selected from one or more of the group consisting of flavin mononucleotide (FMN), flavin adenine dinucleotide (FAD), riboflavin (also known as 7,8-dimethyl-10-((2R,3R,4S)-2,3,4,5-tetrahydroxypentyl) benzo [g] pteridine-2,4 (3H,10H)-dione or lactoflavin) and riboflavin derivatives such as riboflavin-5′-monophosphate, riboflavin-5′-monobutyrate and riboflavin-5′-monopalmitate.

In another separate aspect, the vitamin B3 may be in the form selected from one or more of the group consisting of niacin, and nicotinamide and salts and esters thereof.

In another separate aspect, the vitamin B6 may be in the form selected from one or more of the group consisting of pyridoxine, 3-hydroxy-4,5-bis(hydroxymethyl)2-methylpyridine, 5′-deoxypyridoxal, 2-demethylpyridoxal(2-norpyridoxal), 2-propyl-2-norpyridoxal (2′-ethylpyridoxal), 6-methylpyridoxal, 2′-hydroxypyridoxal (2-hydroxymethyl-2-demethylpyridoxal or 2-hydroxymethyl-2-norpyridoxal), 4′-deoxypyridoxine 5′-phosphate, 5′-methylpyridoxal-5′-phosphate, pyridoxal N-oxide 5′-phosphate, Pyridoxal, Pyridoxamine, Pyridoxine-5′-phosphate (PNP), pyridoxal-5′-phosphate (PLP) and pyridoxamine-5′-phosphate (PMP), and their salts and chelates thereof.

In another separate aspect, the vitamin B9 may be in the form selected from one or more of the group consisting of folic acid, folinic acid, folacin, metafolin, and/or one or more natural isomers of folate including (6S)-tetrahydrofolic acid or a polyglutamyl derivative thereof, (6S,R)-tetrahydrofolic acid or a polyglutamyl derivative thereof, 5-methyl-(6S)-tetrahydrofolic acid or a polyglutamyl derivative thereof, 5-methyl-(6S,R)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof and their salts and esters thereof.

In another separate aspect, the vitamin B12 may be in the form selected from one or more of the group consisting of cobalamin, methylcobalamin, 5′-deoxyadenosylcobalamin, cyanocobalamin, hydroxycobalamin and mecobalamin.

In another separate aspect, the iron may be in the form selected from one or more of the group consisting of elemental iron, in the form of a salt, chelated form, non-chelated form, chelated to an amino acid, carbonyl iron, ferrous gluconate, ferrous fumarate, polysaccharide iron complex, elemental polysaccharide iron, polysaccharide iron, ferrous (II)-bis-glycinate chelate, ferrous asparto glycinate, ferrous bisglycinate, ferrous bisglycinate hydrochloride, ferrous bisglycinate, elemental ferrous bisglycinate, ferrous sulfate, ferronyl (micronized), as Iron Aid, iron protein succinylate, carbonyl iron, Sumalate iron, Heme iron complex, as Ferrochel amino acid chelate, Heme iron polypeptide as Proferrin-bovine source, as heme iron polypeptide (bovine source) as sodium iron EDTA (Ferrazone), ferric ammonium citrate, elemental iron, and ferric pyrophosphate.

In another separate aspect, the iodine may be in the form selected from one or more of the group consisting of iodide, elemental iodine, iodized salt, Lugol's iodine, sodium iodide, potassium iodide, potassium iodate, nascent iodine, and Nano-Colloidal Detoxified Iodine.

In another separate aspect, the magnesium may be in the form selected from one or more of the group consisting of elemental magnesium, in the form of a salt, in a chelated form, in a non-chelated form, magnesium acetate, magnesium carbonate, magnesium gluconate, magnesium chloride, magnesium citrate, magnesium silicate, magnesium stearate, magnesium sulfate, magnesium oxide, and magnesium chelated to an amino acid.

In another separate aspect, the zinc may be in the form selected from one or more of the group consisting of elemental zinc, in the form of a salt, in a chelated form, in a non-chelated form, zinc acetate, zinc gluconate, zinc picolinate, zinc sulfate and zinc oxide.

In another separate aspect, the copper may be in the form selected from one or more of the group consisting of elemental copper, in the form of a salt, in a chelated form, in a non-chelated form, cupric oxide, copper sulfate, copper gluconate, copper citrate, cupric acetate, and alkaline copper carbonate.

In another separate aspect, the source of omega-3 fatty acids may be selected from one or more of the group consisting of: one or more of animal, fish, plants, algae or microorganism production.

In another separate aspect, the source of the omega-3 fatty acids may be algal oil from one or more algae selected from the group consisting of: Schizochytrium sp, Crypthecodinium cohnii, Ulkenia sp. SAM2179, Schizochytrium linacinum strain SC-1

In another separate aspect, the laxative comprises docusate sodium. The laxative may comprise at least about 50 mg of the docusate sodium.

In another embodiment, a kit is provided. The kit comprises a first composition and an adjuvant composition. The first composition comprises vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids and one or more pharmaceutically-acceptable carriers. The adjuvant composition may be formulated to mitigate at least one undesired side effect associated with the administering of the first composition to the patient. The first composition may comprise at least about 90 mg of iron. The iron may be provided in the form of a polysaccharide iron complex.

In one separate aspect, one or both of a first outer coating may be provided to surround the first composition and an adjuvant outer coating to surround the adjuvant composition. The one or both of the first outer coating and the adjuvant outer coating may be separately selected from the group consisting of: a hard shell, a soft shell, and a coating. The soft shell may be a gelatin shell. Alternatively, the coating may be selected from the group consisting of: a polymeric coating, an enteric coating and a sugar coating.

In another separate aspect, the at least one undesired side effect may be constipation. The adjuvant composition may comprise one or a combination of fiber and a laxative. The laxative may be a stool softener and the stool softener may be docusate sodium.

In a further embodiment, a nutritional composition comprises an outer coating encapsulating a filler composition comprising vitamin A, at least 60 mg of vitamin C, vitamin D, vitamin E, vitamin B1, vitamin B2, vitamin B3, vitamin B6, vitamin B9, vitamin B12, at least about 90 mg iron, iodine, magnesium, zinc, copper, a source of omega-3 fatty acids, and one or more pharmaceutically-acceptable carriers. The outer coating may be selected from the group consisting of: a hard shell, a soft shell and a coating.

In one separate aspect, the nutritional composition may further comprise an adjuvant composition comprising one or a combination of a fiber and a laxative.

In another separate aspect, the adjuvant may comprise a laxative and the laxative may be docusate sodium.

In yet a further embodiment, a method is provided. The method comprises administering one or both of a first nutritional composition and an adjuvant composition to a patient in need thereof. The first nutritional composition may comprise vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids. The adjuvant composition may be formulated to mitigate at least one undesired side effect associated with the administering of the first nutritional composition to the patient.

In accordance with one separate aspect, the patient in need thereof is suffering from one or more disease states associated with a nutritional deficiency.

In another separate aspect, the patient may be a female human, and the administering may be performed before pregnancy, during pregnancy, after pregnancy, while breast-feeding, or a combination thereof.

In another separate aspect, the first nutritional composition comprises at least 90 mg of iron.

In another separate aspect, the adjuvant composition may be a laxative.

In another separate aspect, the laxative may be docusate sodium.

In another aspect, the administering may be co-administering both of the first nutritional composition and the adjuvant composition to the patient.

### DETAILED DESCRIPTION OF THE EXEMPLARY EMBODIMENTS

It is understood that the present invention is not limited to the particular methodologies, protocols, fillers, and excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention. All references cited herein are incorporated by reference herein in their entirety.

The term “disease state” as used herein, may comprise any state in which one or more organs or components of an organism malfunction. The term “disease state” may refer to any deterioration of any component of a patient's body and specifically a human patient's body. The term “disease state” may refer to any deficiency of any compound necessary for the maintenance or function of any component of any organism. The term “disease state” may refer to any condition in which a body contains toxins, produced by microorganisms that infect the body or by body cells through faulty metabolism or absorbed from an external source. A “disease state” may also include adverse states caused by any diet, any virus, fungi or any bacteria. A “disease state” may also include disorders associated with pregnant females such as, for example, osteomalacia and preeclampsia, and disorders associated with a fetus such as, for example, neural tube defects and various fetal abnormalities. A “disease state” may also include any pulmonary disorder such as, for example, bronchitis, bronchiectasis, atelectasis, pneumonia, diseases caused by inorganic dusts, diseases caused by organic dusts, any pulmonary fibrosis, and pleurisy. A “disease state” may also include any hematological/oncological disorder such as, for example, anemia, hemophilia, leukemia, and lymphoma. A “disease state” may also include any cancer such as, for example, breast cancer, lung cancer, prostate cancer, pancreatic cancer, liver cancer, stomach cancer, testicular cancer, ovarian cancer, skin cancer, cancer of the brain, cancer of the mouth, cancer of the throat, and cancer of the neck. A “disease state” may also include any disorder of the immune system such as, for example, acquired immune deficiency syndrome (AIDS), AIDS-related complex, infection by any strain of any human immunodeficiency virus (HIV), and other viruses or pathogens such as bacteria, fungi and parasites. A “disease state” may also include any cardiovascular disorder such as, for example, arterial hypertension, orthostatic hypotension, arteriosclerosis, coronary artery disease, cardiomyopathy, any arrhythmia, any valvular heart disease, endocarditis, pericardial disease, any cardiac tumor, any aneurysm, and any peripheral vascular disorder. A “disease state” may also include any hepatic/biliary disorder such as, for example, jaundice, hepatic steatosis, fibrosis, cirrhosis, hepatitis, any hepatic granuloma, any liver tumor, cholelithiasis, cholecystitis, and choledocholithiasis. A “disease state” may also include a viral infection such as from HIV, herpes virus (HSV-1 and HSV-2), the virus that causes vesicular stomatitis (VSV), measles virus, herpes viridae, human lymprotropic visusess, vesicular stomatitis virus, visna virus, cytomegalovirus, Epstein-Barr virus, influenza virus, pneumonovirus, Sarcoma virus, Syncitial virus and Rubeola virus. A “disease state” may also include a fungal infection such as from Candida albicans and Giardia lamblia. A “disease state” may also include a bacterial infection such as from Staphylococcus, Corynebacerium, Bacillus, Listeria and Streptococcus bacteria, and include species such as Staphylococcus aureus, bacillus anthracis, Helicobacter pylori and, Listeria monocytogenes, and Streptococus agalactiae.

The term “patient,” as used herein, comprises any and all organisms and includes the term “subject.” “Patient” may refer to a human or any other animal. “Patient” may also refer to a fetus.

The phrase “co-administration” refers to administration of two or more compositions to a patient together, which includes administration at about the same time or within a certain specific or desired time.

The phrase “chewable form” refers to any relatively soft compositions that are chewed in the mouth after oral administration, may have a pleasant taste and mouthfeel, and may quickly break into smaller pieces and may begin to dissolve after chewing such that they can be swallowed substantially as a solution.

The phrase “dissolvable form” refers to any compositions that dissolve into a solution in the mouth. Such compositions, in one embodiment, may dissolve within about 60 seconds or less after placement in the mouth without any chewing.

The term “mouthfeel” refers to non-taste-related aspects of the pleasantness experienced by a person while chewing or swallowing a nutritional supplement. Aspects of mouthfeel include, for example and without limitation, the hardness and brittleness of a composition, whether the composition is chewy, gritty, oily, creamy, watery, sticky, easily dissolved, astringent, effervescent, and the like, and the size, shape, and form of the composition (tablet, powder, gel, etc.).

The phrase “pharmaceutically acceptable,” as used herein, refers to those compounds, materials, compositions and/or dosage forms which are, within the scope of sound pharmaceutical/medical judgment, suitable for use in contact with the tissues of human beings and animals without excessive toxicity, irritation, allergic response, or other problem or complication, commensurate with a reasonable benefit/risk ratio. Thus, the phrase “pharmaceutically acceptable carriers,” as used herein, refers to such suitable compounds and materials defined above that may be added to the dosage form to assist in satisfactory processing of the dosage form or provide desirable physical characteristics to the dosage form. For example, “pharmaceutically acceptable carriers” may include, but is not limited to, binders, diluents, lubricants, glidants, colorants, emulsifiers, disintegrants, starches, water, oils, alcohols, preservatives, and sugars. In another example, “pharmaceutically acceptable carriers” refers to dosage forms such as capsules, caplets, soft shell capsules, such as gel-caps used with, for example, compositions comprising or consisting of omega-3 fatty acids such as docosahexaenoic acid (DHA). Thus, “pharmaceutically acceptable carriers” in gel-caps may be in for example, liquid or oil form, and may include a filler or other appropriate liquid vehicle and may be used with omega-3 fatty acids such as docosahexaenoic acid (DHA) and their equivalents. Exemplary “pharmaceutically acceptable carriers” suitable for use in connection with the dosage forms described herein can be found in REMINGTON, THE SCIENCE AND PRACTICE OF PHARMACY (22^{th} ed 2012).

The phrase “swallowable form” refers to any compositions that typically do not or are not configured to readily dissolve when placed in the mouth and may be swallowed whole, preferably without any, or with minimal, chewing or discomfort. Such compositions, in one embodiment, may have a shape containing no sharp edges and a smooth, uniform and substantially bubble free outer coating.

The term “dosage form,” as used herein, may be the form in which the dose is to be administered to the subject or patient. The drug or supplement is generally administered as part of a formulation that includes nonmedical agents. The dosage form has unique physical and pharmaceutical characteristics. Dosage forms, for example, may be solid, liquid or gaseous. “Dosage forms,” may include for example, a capsule, tablet, caplet, a soft shell capsule, such as a gel caplet (gel-cap), syrup, a liquid composition, a powder, a concentrated powder, a concentrated powder admixed with a liquid, a chewable form, a swallowable form, a dissolvable form, an effervescent, a granulated form, and an oral liquid solution. In a specific embodiment, the dosage form may be a gel-cap.

The term “substantially free of added” as used herein, means free from therapeutically effective amounts of compounds when administered in suggested doses, but may include trace amounts of compounds in non-therapeutically effective amounts. For example, one embodiment of a composition that included an inactive ingredient that is a salt or compound including a mineral would still be substantially free of added minerals. For example, trace amounts of titanium dioxide may be provided. Titanium dioxide which is an effective opacifier in powder form, where it is employed as a pigment to provide whiteness and opacity to numerous pharmaceutical products.

As used herein, the terms “inactive,” “inert,” “excipient,” and/or “formulatory” refer to any compound that is an inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

By “active ingredient,” then, includes any compound intended to furnish pharmacological activity or other direct effect in the diagnosis, cure, mitigation, treatment and/or prevention of disease or a condition. See 21 C.F.R. 210.3(b)(7). Further, “active ingredients” include those compounds of the composition that may undergo chemical change during the manufacture of the composition and be present in the final composition in a modified form intended to furnish an activity or effect. Id. These include the vitamins, minerals and nutrients of the compositions and kits.

The term “administrable” defines a composition that is able to be given to a patient. Likewise, “administering” refers to the act of giving a composition to a patient or otherwise making such composition available to a patient or the patient taking a composition.

As used herein, the term “about,” when located before a dosage amount or dosage range of a specific ingredient, refers to an amount or range closely above and/or closely below the stated amount or range that does not manifestly alter the therapeutic effect of the specific ingredient from the stated amount or range and is meant to encompass at least all equivalents of that amount. Thus, the term “about” before a specific value may define a range from about the specific value minus at least 10% or at least 20% to the specific value plus at least 10% or at least 20% of the specific value. For example, “about 50” may define a range from 45 to 55 or a range from 40-60.

The term “prenatal” supplementation includes optimizing specific nutrients before, during, and after the physiological processes of pregnancy and lactation, which can have profound, positive, and comprehensive impacts on the overall wellness of the developing and newborn child as well as on the safety and health of the mother.

Proper nutrition is essential for maintaining health and preventing diseases. Adequate nutrition is especially critical during, for example, nutritionally volatile or physiologically stressful periods such as those including, by way of example and without limitation, pregnancy, lactation, or any disease state. Vitamin and mineral needs are almost universally increased throughout these periods. Increased needs during physiologically stressful states such as pregnancy, lactation or disease state may result from elevated metabolic demand, increased plasma volume, increased quantities of circulating red blood cells, decreased concentrations of nutrients, and decreased concentrations of nutrient-binding proteins such as, for example and without limitation, serum-ferritin, maltose-binding protein, lactoferrin, calmodulin, tocopheryl binding protein, riboflavin binding protein, retinol binding protein, transthyretin, high density lipoprotein-apolipoprotein A1, folic acid binding protein, and 25-hydroxyvitamin D binding protein. Lapido, 72 (Supp.) AMER. J. CLIN. NUTR. 280S-90S (2000).

Optimizing specific nutrients before, during, and after the physiological processes of pregnancy and lactation can have profound, positive, and comprehensive impacts on the overall wellness of the developing and newborn child as well as on the safety and health of the mother. Black, 85 (Supp.) BRIT. J. NUTR. S193-97 (2001); Scholl et al., 146 AMER. J. EPIDEM. 134-41 (1997). Nutrients provided to a mother reach the fetus. Specifically, it is established that substrates for growth and development, for example, circulate within the same pathways that carry drugs to and waste products from the fetus. Exchanges of material between mother and fetus occur primarily in the placenta, where villi containing fetal capillaries protrude into sinuses (intervillous spaces). Maternal arterial blood spurts into these spaces, then drains into maternal uterine veins to be returned to the maternal systemic circulation. Solutes in maternal blood cross the epithelial cells and connective tissue of the villi and the endothelium of the fetal capillaries; these solutes are then carried to the fetus by placental veins, which converge into the umbilical vein. THE MERCK MANUAL OF DIAGNOSIS AND THERAPY, (19th ed. 2011). The compositions, kits and methods disclosed herein may thus provide the means to optimize good health by utilizing vitamin, mineral, and nutritional supplementation. The compositions, kits and methods disclosed herein may be administered to or directed to a subject such as a human or any other organism.

The compositions, kits and methods disclosed herein may include vitamin A. Vitamin A is involved in physiological processes that result in cellular differentiation, cellular maturity, and cellular specificity. Thus, vitamin A is an important component of a nutritional supplement for subjects in physiologically stressful states, such as those caused by pregnancy, lactation or disease state. Zile et al., 131(3) J. NUTR. 705-08 (2001). Care should be taken, however, to avoid excess. Indeed, supplemental vitamin A ingestion during pregnancy has been shown in some studies to be teratogenic or deforming to human and animal embryos. G B Mulder et al., 62(4) TERATOLOGY 214-26 (2000). In one embodiment, vitamin A may be in a form that is a precursor (pro-vitamin) or metabolite of vitamin A that provides similar nutritional value as vitamin A. For example, the pro-vitamin A carotenoid, may be beta carotene. Beta carotene is converted to other forms of vitamin A, specifically retinol, within the body as needed, thereby avoiding the risk of retinol toxicity. Mayne, FASEB J 10:690-701 (1996). In a specific embodiment, vitamin A may be in one or more of the forms of retinol acetate (also known as retinyl acetate or vitamin A acetate), retinol (vitamin A alcohol), retinol palmitate (also known as retinyl palmitate or vitamin A palmitate), retinoic acid (tretinoin), retinal, beta-cryptoxanthin, alpha-carotene, beta-carotene, gamma-carotene, and provitamin A carotenoids.

In a specific embodiment, vitamin A may be in the form of beta carotene as beta carotene also has powerful anti-oxidant properties. Antioxidants are important during physiologically stressful events for numerous reasons. For example, lipid peroxidation has been associated with over 200 disease processes. Rock et al., 96(7) J. AMER. DIET. ASSOC. 693-702 (1996). Antioxidants are especially important during pregnancy because in the first trimester, establishment of blood flow into the intervillous space is associated with a burst of oxidative stress. The inability to mount an effective antioxidant defense against this burst results in early pregnancy loss. Myatt & Cui, 122, HISTOCHEM. CELL BIOL., 369-82 (2004). Further, oxidative stress has been implicated in the pathophysiology of preeclampsia, a toxemia of pregnancy. Llurba et al., 37(4) FREE RADIC. BIOL. MED. 557-70 (2004). Finally, oxidative stress during pregnancy plays an important role in fetal growth, and healthy antioxidant levels are positively correlated with birth weight and length. Myatt & Cui; Lee et al., 58 EUR. J. CLIN. NUTR., 481-87 (2004).

In a specific embodiment of the compositions, kits and methods, vitamin A may be included in amounts ranging from about 550 IU to about 1650 IU. In another specific embodiment, vitamin A may be included in amounts ranging from about 880 IU to about 1320 IU. In another specific embodiment, vitamin A may be included in amounts ranging from about 990 IU to about 1210 IU. In another embodiment, vitamin A may be included in an amount of about 1100 IU.

In another specific embodiment, vitamin A may be included in specific ranges or amounts for each specific form. When provided in their specific forms, the provided numerical range or amount includes the amounts of the specific form and/or compounds that are equivalent to the specific form. For example, vitamin A may be in the form of beta carotene and may be included in the amount of about 1100 IU. Accordingly, in this example, “beta carotene in the amount of about 1100 IU” would include 1000 IU of beta carotene and/or its equivalents and would, for example, include a product having 1100 IU retinol acetate instead of beta carotene.

In another specific embodiment of the compositions, kits and methods, vitamin A in the form of beta carotene may be included in amounts ranging from about 550 IU to about 1650 IU. In another specific embodiment, vitamin A in the form of beta carotene may be included in amounts ranging from about 880 IU to about 1320 IU. In another specific embodiment, vitamin A in the form of beta carotene may be included in amounts ranging from about 990 IU to about 1210 IU. In another embodiment, vitamin A in the form of beta carotene may be included in an amount of about 1100 IU. In another specific embodiment of the compositions, kits and methods, vitamin A may be included in the form of beta carotene and one or more forms of vitamin A. In a specific embodiment, the compositions, kits and methods may include beta carotene and retinol. In another embodiment, the compositions, kits and methods may include beta carotene and retinol acetate.

In another embodiment, vitamin A may be present in an amount determined by a measure of mass, as opposed to International Units. One International Unit (IU) of vitamin A is defined as the biological equivalent of about 0.6 μg of beta carotene, or about 0.3 μg of retinol. See REMINGTON, THE SCIENCE AND PRACTICE OF PHARMACY (22^{th} ed 2012. Accordingly, 550 IU to about 1650 IU is the biological equivalent of about 330 μg to about 990 μg. In another example, about 880 IU to about 1320 IU is the biological equivalent of about 528 μg to about 792 μg. In another example, about 990 IU to about 1210 IU is the biological equivalent of about 594 μg to about 726 μg. In another example, 1100 IU is the biological equivalent of about 660 μg.

In a further embodiment, vitamin A may be present in the nutritional composition in any one or a combination of the forms disclosed herein in an amount of at least about 500 IU, at least about 550 IU, at least about 600 IU, at least about 650 IU, at least about 700 IU, at least about 750 IU, at least about 800 IU, at least about 850 IU, at least about 900 IU, at least about 950 IU, at least about 1000 IU, at least about 1050 IU, at least about 1100 IU, at least about 1150 IU, at least about 1200 IU, at least about 1250 IU, at least about 1300 IU, at least about 1350 IU, at least about 1400 IU, at least about 1450 IU, at least about 1500 IU, at least about 1550 IU, at least about 1600 IU, at least about 1650 IU, at least about 1700 IU, at least about 1750 IU, at least about 1800 IU, at least about 1850 IU, at least about 1900 IU, at least about 1950 IU, at least about 2000 IU, at least about 2050 IU, at least about 2100 IU, at least about 2150 IU, at least about 2200 IU, at least about 2250 IU, at least about 2300 IU, at least about 2350 IU, at least about 2400 IU, at least about 2450 IU and at least about 2500 IU. Vitamin A may be present in the nutritional composition in a range of between and including any two of the foregoing values. In embodiments where vitamin A is present in a combination of forms, each constituent form may be present in a relative amount of about 10%, about 20%, about 30%, about 40%, about 50%, about 60%, about 70%, about 80%, about 90% of any one of the foregoing amounts for vitamin A.

The compositions, kits and methods may comprise or use one or more B-complex vitamins. This class of vitamins comprises water-soluble nutrients generally not stored in the body. They play roles in a variety of biological processes critical to the health of pregnant women, lactating women, and fetuses such as, for example, the metabolism of homocysteine. The B-complex vitamins that may be included in the compositions, kits and methods comprise one or more of vitamin B1, vitamin B2, vitamin B3, vitamin B6, vitamin B9 and vitamin B12.

The compositions, kits and methods may comprise or use vitamin B1. Vitamin B1 plays a role in carbohydrate metabolism and neural function. It is a coenzyme for the oxidative decarboxylation of alpha-ketoacids (e.g., alpha-ketoglutarate and pyruvate) and for transketolase, which is a component of the pentose phosphate pathway. NATIONAL RESEARCH COUNCIL, RECOMMENDED DIETARY ALLOWANCES, page 125 (10th ed. 1989) (hereinafter “RDA”). In another specific embodiment, vitamin B1 may be in one or more of the forms of thiamine, thiamine monophosphate, thiamine diphosphate, thiamine triphosphate, acetiamine, allithiamine, prosultiamine and S-acyl derivatives of thiamine such as benfotiamine, fursultiamine and salts and esters thereof.

In another specific embodiment, vitamin B1 may be included in amounts ranging from about 0.8 mg to about 2.4 mg. In another specific embodiment, vitamin B1 may be included in amounts ranging from about 1.3 mg to about 1.9 mg. In another specific embodiment, vitamin B1 may be included in amounts ranging from about 1.4 mg to about 1.75 mg. In another embodiment, vitamin B1 may be included in an amount of about 1.6 mg.

In another specific embodiment, vitamin B1 may be included in specific ranges or amounts for each specific form. When provided in their specific forms, the provided numerical range or amount includes the amounts of the specific form and/or compounds that are equivalent to the specific form. For example, vitamin B1 may be in the form of thiamine mononitrate and may be included in the amount of about 1.6 mg. Accordingly, in this example, “thiamine mononitrate in the amount of about 1.6 mg” would include 1.6 mg of thiamine mononitrate and/or its equivalents and would, for example, include a product having 1.6 mg allithiamine instead of thiamine mononitrate.

In a further embodiment, vitamin B1 may be present in the nutritional composition in any one or a combination of forms disclosed herein an amount of at least about 0.5 mg, at least about 0.6 mg, at least about 0.7 mg, at least about 0.8 mg, at least about 0.9 mg, at least about 1.0 mg, at least about 1.1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, and at least about 3.0 mg. Vitamin B1 may be present in the nutritional composition in a range of between and including any two of the foregoing values. In embodiments where vitamin B1 is present in a combination of forms, each constituent form may be present in a relative amount of about 10%, about 20%, about 30%, about 40%, about 50%, about 60%, about 70%, about 80%, about 90% of any one of the foregoing amounts for vitamin B1.

The compositions, kits and methods may comprise or use vitamin B2. Vitamin B2 is a component of two flavin coenzymes, flavin mononucleotide (FMN) and flavin adenine dinucleotide (FAD). These flavoenzymes are involved in a number of oxidation-reduction reactions including the conversion of pyridoxine and niacin. RDA, supra at 132. Flavoenzymes also play a role in a number of metabolic pathways such as amino acid deamination, purine degradation and fatty acid oxidation and thus help to maintain carbohydrate, amino acid and lipid metabolism.

In a specific embodiment, vitamin B2 may be in one or more of the forms of flavin mononucleotide (FMN), flavin adenine dinucleotide (FAD), riboflavin (also known as 7,8-dimethyl-10-((2R,3R,4S)-2,3,4,5- tetrahydroxypentyl) benzo [g] pteridine-2,4 (3H,10H)-dione or lactoflavin) and riboflavin derivatives such as riboflavin-5′-monophosphate, riboflavin-5′-monobutyrate and riboflavin-5′-monopalmitate. In a specific embodiment, vitamin B2 may be included in the form of riboflavin.

In another specific embodiment, vitamin B2 may be included in amounts ranging from about 0.9 mg to about 2.7 mg. In another specific embodiment, vitamin B2 may be included in amounts ranging from about 1.5 mg to about 2.2 mg. In another specific embodiment, vitamin B2 may be included in amounts ranging from about 1.6 mg to about 2 mg. In another embodiment, vitamin B2 may be included in an amount of about 1.8 mg.

In another specific embodiment, vitamin B2 in the form of riboflavin may be included in amounts ranging from about 0.9 mg to about 2.7 mg. In another specific embodiment, vitamin B2 in the form of riboflavin may be included in amounts ranging from about 1.5 mg to about 2.2 mg. In another specific embodiment, vitamin B2 in the form of riboflavin may be included in amounts ranging from about 1.6 mg to about 2 mg. In another embodiment, vitamin B2 in the form of riboflavin may be included in an amount of about 1.8 mg.

In another specific embodiment, vitamin B2 may be included in specific ranges or amounts for each specific form. When provided in their specific forms, the provided numerical range or amount includes the amounts of the specific form and/or compounds that are equivalent to the specific form. For example, vitamin B2 may be in the form of riboflavin and may be included in the amount of about 1.8 mg. Accordingly, in this example, “riboflavin in the amount of about 1.8 mg” would include 1.8 mg of riboflavin and/or its equivalents and would, for example, include a product having 1.8 mg flavin mononucleotide instead of riboflavin.

In a further embodiment, vitamin B2 may be present in the nutritional composition in any one or a combination of forms disclosed herein an amount of at least about 0.5 mg, at least about 0.6 mg, at least about 0.7 mg, at least about 0.8 mg, at least about 0.9 mg, at least about 1.0 mg, at least about 1.1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, and at least about 3.5 mg. Vitamin B2 may be present in the nutritional composition in a range of between and including any two of the foregoing values. In embodiments where vitamin B2 is present in a combination of forms, each constituent form may be present in a relative amount of about 10%, about 20%, about 30%, about 40%, about 50%, about 60%, about 70%, about 80%, about 90% of any one of the foregoing amounts for vitamin B2.

The compositions, kits and methods may comprise or use vitamin B3. Vitamin B3, or “niacin” is the common name for two compounds: nicotinic acid (also called niacin) and niacinamide (also called nicotinamide). Vitamin B3 is particularly important for maintaining healthy levels and types of fatty acids. It is also required for the synthesis of pyroxidine, riboflavin, and folic acid. RDA, supra at 137. Administration of vitamin B3 also may effect a reduction in total cholesterol (LDL) and very low density lipoprotein (VLDL) levels and an increase in high density lipoprotein (HDL) cholesterol levels. Nicotinamide adenine dinucleotide (NAD) and NAD phosphate (NADP) are active coenzymes of niacin. These coenzymes are involved in numerous enzymatic reactions such as glycolysis, fatty acid metabolism, and steroid synthesis. Henkin et al., 91 AM. J. MED. 239-46 (1991). In a specific embodiment, vitamin B3 may in the forms of niacin (nicotinic acid or pyridine-3-carboxylic acid), and nicotinamide (niacinamide) and salts and esters thereof. In a specific embodiment, vitamin B3 may be included in the form of nicotinamide. In another specific embodiment, an equivalent molar amount of niacin may be included.

In another specific embodiment, vitamin B3 may be included in amounts ranging from about 7.5 mg to about 22.5 mg. In another specific embodiment, vitamin B3 may be included in amounts ranging from about 12 mg to about 18 mg. In another specific embodiment, vitamin B3 may be included in amounts ranging from about 13.5 mg to about 16.5 mg. In another embodiment, vitamin B3 may be included in an amount of about 15 mg.

In another specific embodiment, vitamin B3 may be included in specific ranges or amounts for each specific form. When provided in their specific forms, the provided numerical range or amount includes the amounts of the specific form and/or compounds that are equivalent to the specific form. For example, vitamin B3 may be in the form of nicotinamide and may be included in the amount of about 15 mg. Accordingly, in this example, “nicotinamide in the amount of about 15 mg” would include 15 mg of nicotinamide and/or its equivalents and would, for example, include a product having 15 mg niacin instead of nicotinamide.

In another specific embodiment, vitamin B3 in the form of nicotinamide may be included in amounts ranging from about 7.5 mg to about 22.5 mg. In another specific embodiment, vitamin B3 in the form of nicotinamide may be included in amounts ranging from about 12 mg to about 18 mg. In another specific embodiment, vitamin B3 in the form of nicotinamide may be included in amounts ranging from about 13.5 mg to about 16.5 mg. In another embodiment, vitamin B3 in the form of nicotinamide may be included in an amount of about 15 mg.

In a further embodiment, vitamin B3 may be present in the nutritional composition in any one or a combination of forms disclosed herein an amount of at least about 5 mg, at least about 5.5 mg, at least about 6 mg, at least about 6.5 mg, at least about 7 mg, at least about 7.5 mg, at least about 8 mg, at least about 8.5 mg, at least about 9 mg, at least about 9.5 mg, at least about 10 mg, at least about 10.5 mg, at least about 11 mg, at least about 11.5 mg, at least about 12 mg, at least about 12.5 mg, at least about 13 mg, at least about 13.5 mg, at least about 14 mg, at least about 14.5 mg, at least about 15 mg, at least about 15.5 mg, at least about 16 mg, at least about 16.5 mg, at least about 17 mg, at least about 17.5 mg, at least about 18 mg, at least about 18.5 mg, at least about 19 mg, at least about 19.5 mg, at least about 20 mg, at least about 20.5 mg, at least about 21 mg, at least about 21.5 mg, at least about 22 mg, at least about 22.5 mg, at least about 23 mg, at least about 23.5 mg, at least about 24 mg, at least about 24.5 mg, and at least about 25 mg. Vitamin B3 may be present in the nutritional composition in a range of between and including any two of the foregoing values. In embodiments where vitamin B3 is present in a combination of forms, each constituent form may be present in a relative amount of about 10%, about 20%, about 30%, about 40%, about 50%, about 60%, about 70%, about 80%, about 90% of any one of the foregoing amounts for vitamin B3.

The compositions, kits and methods may comprise or use vitamin B6. The administration of vitamin B6 may reduce the levels of homocysteine. Bostom et al., 49 KIDNEY INT. 147-52 (1996). The active forms of vitamin B6, pyridoxal-5′-phosphate (PLP) and pyridoxamine-5′-phosphate, are coenzymes for numerous enzymes and as such, are important for gluconeogenesis, niacin formation, and erythrocyte metabolism. RDA, supra at 142-43. Vitamin B6 is a coenzyme for both cystathionine synthase and cystathionase, enzymes that catalyze the formation of cysteine from methionine. Homocysteine is an intermediate in this process and elevated levels of plasma homocysteine are recognized as a risk factor for both vascular disease (Robinson et al., 94 CIRCULATION 2743-48 (1996)) and neural tube defects (Locksmith & Duff, 91 OBSTET. GYNECOL. 1027-34 (1998)). In a specific embodiment, vitamin B6 may be included in the forms of pyridoxine, 3-hydroxy-4,5-bis(hydroxymethyl)2-methylpyridine, 5′-deoxypyridoxal, 2-demethylpyridoxal(2-norpyridoxal), 2-propyl-2-norpyridoxal (2′-ethylpyridoxal), 6-methylpyridoxal, 2′-hydroxypyridoxal (2-hydroxymethyl-2-demethylpyridoxal or 2-hydroxymethyl-2-norpyridoxal), 4′-deoxypyridoxine 5′-phosphate, 5′-methylpyridoxal-5′-phosphate, pyridoxal N-oxide 5′-phosphate, Pyridoxal, Pyridoxamine, Pyridoxine-5′-phosphate (PNP), pyridoxal-5′-phosphate (PLP) and pyridoxamine-5′-phosphate (PMP), and their salts and chelates thereof. In a specific embodiment, vitamin B6 may be included in the form of pyridoxine hydrochloride.

In another specific embodiment, vitamin B6 may be included in specific ranges or amounts for each specific form. When provided in their specific forms, the provided numerical range or amount includes the amounts of the specific form and/or compounds that are equivalent to the specific form. For example, vitamin B6 may be in the form of pyridoxine hydrochloride and may be included in the amount of about 2.5 mg. Accordingly, in this example, “pyridoxine hydrochloride in the amount of about 2.5 mg” would include 2.5 mg of pyridoxine hydrochloride and/or its equivalents and would, for example, include a product having 2.5 mg pyridoxamine instead of pyridoxine hydrochloride.

In another specific embodiment, vitamin B6 may be included in amounts ranging from about 1.2 mg to about 3.8 mg. In another specific embodiment, vitamin B6 may be included in amounts ranging from about 2.0 mg to about 3.0 mg. In another specific embodiment, vitamin B6 may be included in amounts ranging from about 2.25 mg to about 2.75 mg. In another embodiment, vitamin B6 may be included in an amount of about 2.5 mg.

In a further embodiment, vitamin B6 may be present in the nutritional composition in any one or a combination of forms disclosed herein an amount of at least about 1 mg, at least about 1.1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, and at least about 4.5 mg. Vitamin B6 may be present in the nutritional composition in a range of between and including any two of the foregoing values. In embodiments where vitamin B6 is present in a combination of forms, each constituent form may be present in a relative amount of about 10%, about 20%, about 30%, about 40%, about 50%, about 60%, about 70%, about 80%, about 90% of any one of the foregoing amounts for vitamin B6.

The compositions, kits and methods may comprise or use vitamin B9. Vitamin B9 is a generic name of a B-vitamin that includes multiple compounds with a general structure. For example, vitamin B9 encompasses the term folate, which itself is the generic name for many different forms of this water-soluble vitamin (vitamin B9), which is essential for DNA synthesis and, hence, cell division. Simpson et al., THE JOURNAL OF MATERNAL-FETAL AND NEONATAL MEDICINE, Micronutrients and women of reproductive potential: required dietary intake and consequences of dietary deficiency or excess. Part I—Folate, Vitamin B12, Vitamin B6, Epub 1-21, (2010). Indeed, folate encompasses numerous compounds that for example, are based on a pteridine ring, an aminobenzoic acid and one or more glutamic acid residues. Id. Folic acid (pteroglutamic acid or PGA) is a synthetic form of folate, and the first folate synthesized and used as a supplement. Id. The term folates may also be used in the generic sense to designate any members of the family of pteroylglutamates, or mixtures of them, having various levels of reduction of the pteridine ring, one-carbon substitutions and numbers of glutamate residues. PURE & APPL. CHEM., IUPAC-IUB Commission on Biochemical Nomenclature (CBN). Nomenclature and Symbols for Folic Acid and Related Compounds. Arch 59, No. 6: 833-836 (1987).

Vitamin B9, however, is not only defined by its structure, but also by its various functions. Indeed, vitamin B9 is essential for DNA synthesis and, hence, cell division and is required metabolically as a coenzyme in one-carbon transfer reactions. Simpson, supra. This vitamin has demonstrated the ability to prevent neural tube defects such as spina bifida caused by disturbed homocysteine metabolism. Vanderput et al., EXP. BIOL. MED. 243-70 (2001); DeFalco et al., 27 CLIN. EXP. OBSTET. GYNECOL. 188-90 (2000); Eskes, 27 CLIN. EXP. OBSTET. GYNECOL. 157-67 (2000); Locksmith & Duff, supra. Folic acid, a commonly used term synonymous with vitamin B9, is known to reduce the risk of multiple diseases. Clinical trials definitively demonstrated the effectiveness of folic acid supplementation in reducing the number of neural tube defects. Simpson et al., THE JOURNAL OF MATERNAL-FETAL AND NEONATAL MEDICINE, Micronutrients and women of reproductive potential: required dietary intake and consequences of dietary deficiency or excess. Part I—Folate, Vitamin B12, Vitamin B6, Epub 1-21, (2010). Indeed, folic acid supplementation in reducing the risk of neural tube defects and other congenital malformations is generally accepted. Pietrzik et al., CLIN PHARMACOKINET 49 (8): 535-548 (2010). Furthermore, evidence is accumulating to support a possible role of folic acid in the reduction in risk of other diseases, including dementia and certain types of cancer. Id. Lastly, folate or folate derivative thereof that increase blood folate levels, thereby reducing homocysteine levels, which is a common way to measure vitamin B9 effectiveness. Id.

Thus, in a specific embodiment, vitamin B9 may include numerous forms. In a specific embodiment, vitamin B9 may be included in the form of folic acid. In another embodiment, vitamin B9 may be included one or more of the forms of folic acid, folacin, metafolin, folate and/or one or more natural isomers of folate including (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof and the salts and esters thereof. In another embodiment, vitamin B9 may be in the form of a folate or folate derivative thereof that is eventually converted to 5-methyl-tetrahydrofolic acid in the body and/or is absorbed into the bloodstream as 5-methyl-tetrahydrofolic acid. Folates, such as folic acid and folate, are eventually absorbed in the body and converted to L-5-methyl-tetrahydrofolic acid. In another embodiment, vitamin B9 may be in the form of a folate or folate derivative thereof that increases blood folate levels, thereby reducing homocysteine levels.

In another embodiment, vitamin B9 may be in the form of folate or reduced folates with various salts. In a specific embodiment, the folate and reduced folate are selected from the group consisting of D-glucosamine-folate, D-galactosamine- folate, D-glucosamine (6R, S)-tetrahydrofolate, D-glucosamine (6S)-tetrahydrofolate, D-glucosamine (6R)-tetrahydrofolate; D-galactosamine (6R, S)-tetrahydrofolate, D-galactosamine (6S)-tetrahydrofolate, D-galactosamine (6R)-tetrahydrofolate; D-glucosamine 5-methyl-(6R, S)-tetrahydrofolate, D-glucosamine 5-methyl-(6S)-tetrahydrofolate, D-glucosamine 5-methyl-(6R)-tetrahydrofolate; D-galactosamine 5-methyl-(6R, S)-tetrahydrofolate, D-galactosamine 5-methyl-(6S)-tetrahydrofolate, and D-galactosamine 5-methyl-(6R)-tetrahydrofolate.

In another specific embodiment, vitamin B9 may be included in amounts ranging from about 0.5 mg to about 1.5 mg. In another specific embodiment, vitamin B9 may be included in amounts ranging from about 0.8 mg to about 1.2 mg. In another specific embodiment, vitamin B9 may be included in amounts ranging from about 0.9 mg to about 1.1 mg. In another embodiment, vitamin B9 may be included in an amount of about 1.0 mg.

In another specific embodiment, vitamin B9 may be included in specific ranges or amounts for each specific form. When provided in their specific forms, the provided numerical range or amount includes the amounts of the specific form and/or compounds that are equivalent to the specific form. For example, vitamin B9 may be in the form folic acid and may be included in the amount of about 1.0 mg. Accordingly, in this example, “folic acid in the amount of about 1.0 mg” would include 1.0 mg of folic acid and/or its equivalents and would, for example, include a product having 1.0 mg 5-methyl-(6S)-tetrahydrofolic acid instead of folic acid.

In a specific embodiment, vitamin B9 may be in the form of folic acid. In another specific embodiment, vitamin B9 in the form of folic acid may be included in amounts ranging from about 0.5 mg to about 1.5 mg. In another specific embodiment, vitamin B9 in the form of folic acid may be included in amounts ranging from about 0.8 mg to about 1.2 mg. In another specific embodiment, vitamin B9 in the form of folic acid may be included in amounts ranging from about 0.9 mg to about 1.1 mg. In another embodiment, vitamin B9 in the form of folic acid may be included in an amount of about 1.0 mg.

In a further embodiment, vitamin B9 may be present in the nutritional composition in any one or a combination of forms disclosed herein an amount of at least about 0.5 mg, at least about 0.6 mg, at least about 0.7 mg, at least about 0.8 mg, at least about 0.9 mg, at least about 1.0 mg, at least about 1.1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, and at least about 3.5 mg. Vitamin B9 may be present in the nutritional composition in a range of between and including any two of the foregoing values. In embodiments where vitamin B9 is present in a combination of forms, for example folic acid and L-methylfolate calcium, each constituent form may be present in a relative amount of about 10%, about 20%, about 30%, about 40%, about 50%, about 60%, about 70%, about 80%, about 90% of any one of the foregoing amounts for vitamin B9. In one specific embodiment, for example, vitamin B9 may be provided in an amount of 0.4 mg folic acid and 0.6 mg L-methylfolate calcium.

The compositions, kits and methods may comprise or use vitamin B12. Vitamin B12 can be converted to the active coenzymes, methylcobalamin and 5′-deoxyadenosylcobalamin. These coenzymes are necessary for folic acid metabolism, conversion of coenzyme A and myelin synthesis. Methylcobalamin also catalyzes the demethylation of a folate cofactor which is involved in DNA synthesis. A lack of demethylation may result in folic acid deficiency. RDA, supra at 159-160. Deoxyadenosylcobalamin is the coenzyme for the conversion of methylmalonyl-CoA to succinyl-CoA, which plays a role in the citric acid cycle. Cobalamin, along with pyridoxine and folic acid, also are implicated in the proper metabolism of homocysteine, a breakdown product of the amino acid methionine, which is correlated with an increased risk of heart disease due to its negative effects on endothelial function. In a specific embodiment, vitamin B12 may be in one or more of the forms of cobalamin, methylcobalamin, 5′-deoxyadenosylcobalamin (adenosylcobalamin or cobamamide), cyanocobalamin, hydroxycobalamin and mecobalamin.

In another specific embodiment, vitamin B12 may be included in amounts ranging from about 6 μg to about 18 μg. In another specific embodiment, vitamin B12 may be included in amounts ranging from about 9.6 μg to about 14.4 μg. In another specific embodiment, vitamin B12 may be included in amounts ranging from about 10.8 μg to about 13.2 μg. In another embodiment, vitamin B12 may be included in an amount of about 12 μg.

In another specific embodiment, vitamin B12 may be included in specific ranges or amounts for each specific form. When provided in their specific forms, the provided numerical range or amount includes the amounts of the specific form and/or compounds that are equivalent to the specific form. For example, vitamin B12 may be in the form cyanocobalamin and may be included in the amount of about 12 μg. Accordingly, in this example, “cyanocobalamin in the amount of about 12 μg” would include 12 μg of cyanocobalamin and/or its equivalents and would, for example, include a product having 12 μg methylcobalamin instead of cyanocobalamin.

In a further embodiment, vitamin B12 may be present in the nutritional composition in any one or a combination of forms disclosed herein an amount of at least about 1 mg, at least about 5 μg, at least about 5.5 μg, at least about 6 μg, at least about 6.5 μg, at least about 7 μg, at least about 7.5 μg, at least about 8 μg, at least about 8.5 μg, at least about 9 μg, at least about 9.5 μg, at least about 10 μg, at least about 10.5 μg, at least about 11 μg, at least about 11.5 μg, at least about 12 μg, at least about 12.5 μg, at least about 13 μg, at least about 13.5 μg, at least about 14 μg, at least about 14.5 μg, at least about 15 μg, at least about 15.5 μg, at least about 16 μg, at least about 16.5 μg, at least about 17 μg, at least about 17.5 μg, at least about 18 μg, at least about 18.5 μg, at least about 19 μg, at least about 19.5 μg, and at least about 20 μg. Vitamin B12 may be present in the nutritional composition in a range of between and including any two of the foregoing values. In embodiments where vitamin B12 is present in a combination of forms, each constituent form may be present in a relative amount of about 10%, about 20%, about 30%, about 40%, about 50%, about 60%, about 70%, about 80%, about 90% of any one of the foregoing amounts for vitamin B12.

The compositions, kits and methods may comprise or use vitamin C. The major biochemical role of water-soluble vitamin C is as a co-substrate in metal catalyzed hydroxylations. Like beta carotene, vitamin C has antioxidant properties. It interacts directly with superoxide hydroxyl radicals and singlet oxygen, and also provides antioxidant protection for folate and vitamin E, keeping vitamin E in its most potent form. Vitamin C may afford protective effects against preeclampsia by participating in the scavenging of free radicals. Indeed, significantly lower levels of vitamin C have been observed in preeclamptic women than in controls. Woods et al., 185(1) AM. J. OBSTET. GYNECOL. 5-10 (2001); Kharb, 1 EURO. J. OBSTET. GYNECOL. REPROD. BIOL. 37-39 (2000); Milczarek et al., 210 MOL. CELL. BIOCHEM. 65-73 (2000). Vitamin C also enhances the absorption of iron. RDA, supra at 115. In addition, vitamin C is required for collagen synthesis, epinephrine synthesis, and bile acid formation. Moreover, vitamin C has been implicated in inhibiting atherosclerosis by being present in extracellular fluid of the arterial wall and potentiating nitric oxide activity, thus normalizing vascular function. In a specific embodiment, vitamin C may be included in the forms of ascorbic acid, ascorbates (calcium or sodium ascorbate), dehydroascorbic acid and salts, ascorbyl palmitate, ascorbyl phosphates and salts (such as sodium or magnesium ascorbyl phosphate), ascorbyl tetraisopalmitate, tetrahexyldecyl ascorbate, ascorbyl sulfates and salts, acylated ascorbic acid derivatives (such as 6-O-acyl-2-O-alpha-D-glucopyranosyl-L-ascorbic acids), 6-bromo-6-deoxy-L-ascorbic acid, and ascorbate salts. In a specific embodiment, vitamin C may be included in the form of ascorbic acid.

In another specific embodiment, vitamin C may be included in amounts ranging from about 15 mg to about 45 mg. In another specific embodiment, vitamin C may be included in amounts ranging from about 24 mg to about 36 mg. In another specific embodiment, vitamin C may be included in amounts ranging from about 27 mg to about 33 mg. In another embodiment, vitamin C may be included in an amount of about 30 mg.

In another specific embodiment, vitamin C may be included in specific ranges or amounts for each specific form. When provided in their specific forms, the provided numerical range or amount includes the amounts of the specific form and/or compounds that are equivalent to the specific form. For example, vitamin C may be in the form ascorbic acid and may be included in the amount of about 30 mg. Accordingly, in this example, “ascorbic acid in the amount of about 30 mg” would include 30 mg of ascorbic acid and/or its equivalents and would, for example, include a product having 30 mg ascorbyl palmitate instead of ascorbic acid.

In another specific embodiment, vitamin C in the form of ascorbic acid may be included in amounts ranging from about 15 mg to about 45 mg. In another specific embodiment, vitamin C in the form of ascorbic acid may be included in amounts ranging from about 24 mg to about 36 mg. In another specific embodiment, vitamin C in the form of ascorbic acid may be included in amounts ranging from about 27 mg to about 33 mg. In another embodiment, vitamin C in the form of ascorbic acid may be included in an amount of about 30 mg.

In a further embodiment, vitamin C may be present in the nutritional composition in any of one or a combination of forms disclosed herein in an amount of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, and at least about 200 mg. Vitamin C may be present in the nutritional composition in a range of between and including any two of the foregoing values. In embodiments where vitamin C is present in a combination of forms, each constituent form may be present in a relative amount of about 10%, about 20%, about 30%, about 40%, about 50%, about 60%, about 70%, about 80%, about 90% of any one of the foregoing amounts for vitamin C.

The compositions, kits and methods may comprise or use vitamin D. In another embodiment, the compositions, kits and methods may include a beneficially increased supplementation of vitamin D. Vitamin D is a fat-soluble “hormone like” substance important for the maintenance of healthy bones. This vitamin increases the absorption of calcium and phosphorous from the gastrointestinal tract, and improves mineral resorption into bone tissue. The result of this physiological function is a correlation between adequate systemic levels in pregnancy and a long-lasting reduction in osteoporotic fractures throughout the lifespan of the newborn. M F Holick, “Vitamin D,” in MODERN NUTRITION IN HEALTH AND DISEASE, p. 313, M E Shils, J A Olsen and M. Shikeeds., Plea and Febiger, Philadelphia, Pa. (1994); M K Javaid et al., LANCET 367(9504):36-43 (2006).

Moreover, recent research suggests that vitamin D has more positive physiological effects than previous thought. Bischoff-Ferrari H A, 624 ADV EXP MED BIOL. 55-71 (2008); Holick M F, 357 N. ENG. J. MED. 266-81, (2007); Parikin et al., 89(3) J CLIN ENDOCRINOL METAB. 1196-99 (2004). For example, it has recently been determined that vitamin D also has a role in the enhancement of vascular function, defense against cancer, immuno-competence, blood pressure regulation and possessing the ability to enhance cellular insulin sensitivity in the human body. Due to the additional roles that vitamin D plays in the human body, it has recently been determined that higher daily vitamin D intake beyond current recommendations may be associated with better health outcomes. Bischoff-Ferrari H A, supra. Indeed, studies suggest increasing the serum level of 25-hydroxyvitamin D, a beneficial derivative of vitamin D, to a 30 ng/ml serum range. Id. A 30 ng/ml appears to be the most advantageous serum levels in recent studies reviewing patient bone mineral density (BMD), lower extremity function, dental health, risk of falls, admission to nursing home, fractures, cancer prevention and incident hypertension. Id.

Further, studies suggest that an intake of about 1000 IU of vitamin D3 (cholecalciferol) per day for all adults may bring at least 50% of the population up to the 30 ng/ml serum range for 25-hydroxyvitamin D. Id. Current nutritional supplements, however, do not provide a high enough dosage for obtaining such a high serum level of 25-hydroxyvitamin D. Presently, the suggested daily amount of vitamin D, as stated by the U.S. Dietary Reference Intake for adequate intake (AI) of vitamin D for infants, children and men and women aged 19-50 is 200 IU/day. Adequate intake increases to 400 IU/day for men and women aged 51-70 and up to 600 IU/day past the age of 70. Id. Due to these studies, present nutritional supplements may be insufficient to remedy the current U.S. and global epidemic related to vitamin D deficiency.

Indeed, research findings indicate vitamin D status during pregnancy is more important than previous thought. Vitamin D's role continues to expand in for example, infant immunity, neurodevelopment, birth weight, and incidence of asthma. Growing research findings regarding the importance of this hormone-like compound is due, in large part, to the fact that vitamin D receptors have now been identified on nearly every tissue and cell in the human body. H F DeLuca et al., FASEB J 15:2579-2585 (2001); D. Eyles et al., NEUROSCIENCE 118(3):641-653 (2003); C A Mannion et al., CMAJ 174(9):1273-1277 (2006); B W Hollis et al., CMAJ 174(9):1287-1290 (2006); American Academy of Allergy, Asthma and Immunology Annual Meeting, Miami, Fla. (March 2006). A nutritional supplement that includes a higher dosage amount of vitamin D, as compared to present nutritional supplements and, specifically, prenatal supplements, is therefore currently needed. Thus, one embodiment provides compositions, kits and methods that provide a beneficial increased supplementation of vitamin D, specifically, for example prenatal, pregnant or breast feeding women.

The vitamin D of the compositions, kits and methods may comprise vitamin D. In a specific embodiment, vitamin D may be in one or more the forms of vitamin D3 (also known as calciol or cholecalciferol or colecalciferol), vitamin D2 (also known as calciferol, ergocalciol, ergocalciferol, ercalciol, Deltalin or Viosterol), previtamin D2, ergosterol, calcitriol (also known as 1,25-dihydroxycholecalciferol), 7-dehydrocholesterol, vitamin D1, vitamin D4 (also known as 22-dihydroergocalciferol, 22,23-dihydroercalciol or (24S)-methylcalciol), vitamin D5 (also known as (24S)-Ethylcalciol or sitocalciferol), 7-dehydrositosterol, Lumisterol, 25-hydroxyvitamin D, all steroids that exhibit the biological activity of calciol, 25-fluorocalciol, (3S)-3-amino-3-deoxycalciol, 11α-acetoxycalciol, calcidiol (also known as 25-hydroxycholecalciferol or calcifediol), ercalcitriol, calcitetrol, tacalciol (also known as tachysterol₃), (5E)-isocalciol (also known as isovitamin D3), Dihydroercalciol (also known as dihydrotachysterol3), (1S)-Hydroxycalciol (also known as 1α-hydroxycholecalciferol or alfacaleidol), (24R)-Hydroxycalcidiol (also known as 24(R),25-dihydroxycholecalciferol), Ercalcidiol, Ercalcitriol, Ertacalciol, (5E)-(10S)-10,19-Dihydroercalciol (also known as dihydrotachysterol2), (6Z)-Tacalciol (also known as precalciferol or pre-vitamin D), and (22E)-(24R)-Ethyl-22,23-didehydrocalciol also known as vitamin D6.

In one embodiment, vitamin D may be present in the amount ranging from about 400 IU to about 1600 IU. In another embodiment, vitamin D may be present in the amount ranging from about 750 IU to about 1250 IU. In another embodiment, vitamin D is present in the amount ranging from about 900 IU to about 1100 IU. In another embodiment, vitamin D is present in the amount of about 1000 IU.

In another specific embodiment, vitamin D may be included in specific ranges or amounts for each specific form. When provided in their specific forms, the provided numerical range or amount includes the amounts of the specific form and/or compounds that are equivalent to the specific form. For example, vitamin D may be in the form vitamin D3 and may be included in the amount of about 1000 IU. Accordingly, in this example, “vitamin D3 in the amount of about 30 mg” would include 1000 IU of vitamin D3 and/or its equivalents and would, for example, include a product having 1000 IU mg vitamin D2 instead of vitamin D3.

In another embodiment, the vitamin D of the compositions, kits and methods may be vitamin D3. In the body, vitamin D3 is produced when its precursor is exposed to ultraviolet irradiation (e.g., sunlight) and then hydroxylated in the liver to form 25-hydroxyvitamin D3, the major form of vitamin D in the circulation. This form of the vitamin may be hydroxylated again in the kidney, yielding 1,25 hydroxyvitamin D3, the most potent form of vitamin D. As noted above, vitamin D3 plays a role in the maintenance of calcium and phosphorus homeostasis, but it is also active in cell differentiation and immune function.

In one embodiment, vitamin D in the form of vitamin D3 may be present in the amount ranging from about 400 IU to about 1600 IU. In another embodiment, vitamin D in the form of vitamin D3 may be present in the amount ranging from about 750 IU to about 1250 IU. In another embodiment, vitamin D in the form of vitamin D3 may be present in the amount ranging from about 900 IU to about 1100 IU. In another embodiment, vitamin D in the form of vitamin D3 may be present in the amount of about 1000 IU.

In another embodiment, vitamin D may be present in an amount determined by a measure of mass, as opposed to International Units. One International Unit (IU) of vitamin D is defined as the biological equivalent of about 0.025 μg of vitamin D3. See REMINGTON, THE SCIENCE AND PRACTICE OF PHARMACY (22^{th} ed 2012). Accordingly, 400 IU to about 1600 IU is the biological equivalent of about 10 μg to about 40 μg. In another example, about 750 IU to about 1250 IU is the biological equivalent of about 18.75 μg to about 31.25 μg. In another example, about 900 IU to about 1100 IU is the biological equivalent of about 22.5 μg to about 27.5 μg. In another example, 1000 IU is the biological equivalent of about 25 μg.

In a further embodiment, vitamin D may be present in the nutritional composition in any of one or a combination of forms disclosed herein in an amount of at least about 400 IU, at least about 450 IU, at least about 500 IU, at least about 550 IU, at least about 600 IU, at least about 650 IU, at least about 700 IU, at least about 750 IU, at least about 800 IU, at least about 850 IU, at least about 900 IU, at least about 950 IU, at least about 1000 IU, at least about 1050 IU, at least about 1100 IU, at least about 1150 IU, at least about 1200 IU, at least about 1250 IU, at least about 1300 IU, at least about 1350 IU, at least about 1400 IU, at least about 1450 IU, at least about 1500 IU, at least about 1550 IU, at least about 1600 IU, at least about 1650 IU, at least about 1700 IU, at least about 1750 IU, at least about 1800 IU, at least about 1850 IU, at least about 1900 IU, and at least about 1950 IU. Vitamin D may be present in the nutritional composition in a range of between and including any two of the foregoing values. In embodiments where vitamin D is present in a combination of forms, each constituent form may be present in a relative amount of about 10%, about 20%, about 30%, about 40%, about 50%, about 60%, about 70%, about 80%, about 90% of any one of the foregoing amounts for vitamin D.

The compositions, kits and methods may comprise or use vitamin E. Vitamin E is a fat-soluble vitamin antioxidant found in biological membranes where it protects the phospholipid membrane from oxidative stress. Vitamin E inhibits the oxidation of unsaturated fatty acids by trapping peroxyl free radicals. It is also an antiatherogenic agent, and studies have demonstrated a reduced risk of coronary heart disease with increased intake of vitamin E. Stampfer et al., 328 NEW ENG. J. MED. 1444-49 (1993). In addition, vitamin E, like beta carotene and vitamin C, may afford protective effects against preeclampsia by participating in the scavenging of free radicals. As with vitamin C, significantly lower levels of vitamin E have been observed in preeclamptic women than in controls. Woods et al., AM J OBSTET GYNECOL, 185(1):5-10 (2001); Kharb, EURO. J. OBSTET GYNECOL REPROD BIOL, 1:37-39 (2000); Milczarek et al., MOL CELL BIOCHEM, 210:65-73 (2000). In a specific embodiment, vitamin E may be included in one or more of the forms of alpha, beta, gamma, and delta tocopherols in its natural or synthetic (dl) forms; alpha, beta, gamma, and delta tocotrienols in its natural or synthetic (dl) forms, dl-alpha tocopheryl derivatives such as dl-alpha tocopheryl esters, dl-alpha-tocopheryl acetate or succinate and d-alpha-tocopheryl acetate or dl-alpha tocopheryl phosphates (such as Ester-E®). In a specific embodiment, vitamin E may be included in the form of d-alpha-tocopheryl acetate. In another specific embodiment, vitamin E may be included in the form of an equivalent molar amount of d-alpha tocopheryl succinate.

In another specific embodiment, vitamin E may be included in amounts ranging from about 10 IU to about 30 IU. In another specific embodiment, vitamin E may be included in amounts ranging from about 15 IU to about 25 IU. In another specific embodiment, vitamin E may be included in amounts ranging from about 18 IU to about 22 IU. In another embodiment, vitamin E may be included in an amount of about 20 IU.

In another specific embodiment, vitamin E may be included in specific ranges or amounts for each specific form. When provided in their specific forms, the provided numerical range or amount includes the amounts of the specific form and/or compounds that are equivalent to the specific form. For example, vitamin E may be in the form d-alpha-tocopheryl acetate and may be included in the amount of about 20 IU. Accordingly, in this example, “d-alpha-tocopheryl in the amount of about 20 IU” would include 20 IU of d-alpha-tocopheryl and/or its equivalents and would, for example, include a product having 20 IU alpha-tocotrienol instead of d-alpha-tocopheryl.

In a further embodiment, vitamin E may be present in the nutritional composition in any of one or a combination of forms disclosed herein in an amount of at least about 5 IU, at least about 6 IU, at least about 7 IU, at least about 8 IU, at least about 9 IU, at least about 10 IU, at least about 11 IU, at least about 12 IU, at least about 13 IU, at least about 14 IU, at least about 15 IU, at least about 16 IU, at least about 17 IU, at least about 18 IU, at least about 19 IU, at least about 20 IU, at least about 21 IU, at least about 22 IU, at least about 23 IU, at least about 24 IU, at least about 25 IU, at least about 26 IU, at least about 27 IU, at least about 28 IU, at least about 29 IU, at least about 30 IU, at least about 31 IU, at least about 32 IU, at least about 33 IU, at least about 34 IU, at least about 35 IU, at least about 36 IU, at least about 37 IU, at least about 38 IU, at least about 39 IU, and at least about 40 IU. Vitamin E may be present in the nutritional composition in a range of between and including any two of the foregoing values. In embodiments where vitamin E is present in a combination of forms, each constituent form may be present in a relative amount of about 10%, about 20%, about 30%, about 40%, about 50%, about 60%, about 70%, about 80%, about 90% of any one of the foregoing amounts for vitamin E.

The compositions, kits and methods may comprise or use iron. A primary function of iron is to carry oxygen to bodily tissues via the hemoglobin part of red blood cells. Supplemental intake of iron is critical to preventing anemia, a disorder associated with a variety of physiological states including, for example, pregnancy. Bothwell, 72(Supp.) AM. J. CLIN. NUTR. 257S-64S (2000). Severe anemia may have adverse effects upon a mother and a fetus. Specifically, significant depression of hemoglobin has been associated with poor pregnancy outcome. Black, supra; Sifakis & Pharmakides, 900 ANN. N.Y. ACAD. SCI. 125-36 (2000). The compositions, kits and methods may include iron in one or more of the forms of elemental iron, in the form of a salt, chelated form, non-chelated form, chelated to an amino acid, carbonyl iron, ferrous gluconate, ferrous fumarate, polysaccharide iron complex, elemental polysaccharide iron, polysaccharide iron, ferrous (II)-bis-glycinate chelate, ferrous asparto glycinate, ferrous bisglycinate, ferrous bisglycinate hydrochloride, ferrous bisglycinate, elemental ferrous bisglycinate, ferrous sulfate, ferronyl (micronized), as Iron Aid, iron protein succinylate, carbonyl iron, Sumalate iron, Heme iron complex, as Ferrochel amino acid chelate, heme iron polypeptide as Proferrin-bovine source, as heme iron polypeptide (bovine source) as sodium iron EDTA (Ferrazone), ferric ammonium citrate, elemental iron, and ferric pyrophosphate.

In a specific embodiment, iron may be included in the form of polysaccharide iron complex. In another specific embodiment, iron may be included in the form of an equivalent molar amount of ferrous fumarate. In another specific embodiment, iron may be included in amounts ranging from about 14.5 mg to about 43.5 mg. In another specific embodiment, iron may be included in amounts ranging from about 21.6 mg to about 32.4 mg. In another specific embodiment, iron may be included in amounts ranging from about 26 mg to about 32 mg. In another embodiment, iron may be included in an amount of about 29 mg.

In another specific embodiment, iron may be included in specific ranges or amounts for each specific form. When provided in their specific forms, the provided numerical range or amount includes the amounts of the specific form and/or compounds that are equivalent to the specific form. For example, iron may be in the form polysaccharide iron complex and may be included in the amount of about 29 mg. Accordingly, in this example, “polysaccharide iron complex in the amount of about 29 mg” would include 29 mg of polysaccharide iron complex and/or its equivalents and would, for example, include a product having 29 mg ferrous fumarate instead of polysaccharide iron complex.

In a further embodiment, iron may be present in the nutritional composition in any one or a combination of forms disclosed herein an amount of at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, and at least about 125 mg. Iron may be present in the nutritional composition in a range of between and including any two of the foregoing values. In embodiments where iron present in a combination of forms, each constituent form may be present in a relative amount of about 10%, about 20%, about 30%, about 40%, about 50%, about 60%, about 70%, about 80%, about 90% of any one of the foregoing amounts for iron.

The compositions, kits and methods may comprise or use iodine. Iodine provides nutritional benefits as it is an essential component of the thyroid hormones that are involved in the regulation of various enzymes and metabolic processes, such as thyroxine and triiodothyronine. Thyroid hormones play pivotal roles in metabolism. Consequences of deficiency (hypothyroidism) and excess (hyperthyroidism) are well-recognized clinically. Simpson et al., THE JOURNAL OF MATERNAL-FETAL AND NEONATAL MEDICINE, Micronutrients and women of reproductive potential: required dietary intake and consequences of dietary deficiency or excess. Part II-Vitamin D, Vitamin A, Iron, Zinc, Iodine, Essential Fatty Acids, 1-2, epub online 2010. Indeed, iodine deficiency disorders (IDD) include mental retardation, hypothyroidism, goiter, cretinism, and varying degrees of other growth and developmental abnormalities, which can be a result from inadequate thyroid hormone production from lack of sufficient iodine. Further, iodine is an important element in breast milk for infant nutrition. An adequate concentration of iodine in breast milk is essential to provide for optimal neonatal thyroid hormone stores and to prevent impaired neurological development in breast-fed neonates. In many countries of the world, low iodine content of the breast milk indicates less than optimum maternal and infant iodine nutrition. F. Azizi et al., CLIN ENDOCRINOL; 70(5):803-9 (2009). Iodine deficiency is a major public health problem in nearly all countries, particularly for women during pregnancy and lactation. The National Health and Nutrition Examination survey data also found 14.9% of women aged 15-44 years and 6.9% of pregnant women to have urinary iodine concentrations of only 50 mg/L, indicating iodine intake of less than 100 mg daily. Simpson, supra. The American Thyroid Association thus also recommends that women receive 150 mg iodine supplements daily during pregnancy and during lactation, which is often the upper limit for iodine dosing amounts in prenatal supplements. Id. Regardless of such recommendations, iodine nutrition and supplementation is lacking. For example, in Europe, most women are iodine deficient during pregnancy, with less than 50% receiving iodine supplementation; of 40 countries, only nine met the requirements of iodized salt at the household level to be at least 90% of the DRI. Id. Iodine nutrition of women of childbearing age thus remains inadequate and an area worthy of public health concern. Id. A nutritional supplement that includes a higher dosage amount of iodine, as compared to present nutritional supplements and, specifically, prenatal supplements, is therefore currently needed. Thus, one embodiment, the compositions, kits and methods provide a beneficial increased supplementation of iodine, specifically, for example prenatal, pregnant or breast feeding women.

In a specific embodiment, iodine may be in the forms of elemental iodine, iodized salt, Lugol's iodine, sodium iodide, potassium iodide, potassium iodate, nascent iodine, and Nano-Colloidal Detoxified Iodine. In another specific embodiment, iodine may be present in the amounts ranging from about 75 μg to about 225 μg. In another embodiment, iodine may be present in the amounts ranging from about 120 μg to about 180 μg. In another embodiment, iodine may be present in the amounts ranging from about 135 μg to about 165 μg. In another embodiment, iodine may be present in the amount of about 150 μg.

In another specific embodiment, iodine may be included in specific ranges or amounts for each specific form. When provided in their specific forms, the provided numerical range or amount includes the amounts of the specific form and/or compounds that are equivalent to the specific form. For example, iodine may be in the form potassium iodide and may be included in the amount of about 150 μg. Accordingly, in this example, “potassium iodide in the amount of about 150 μg” would include 150 μg of potassium iodide and/or its equivalents and would, for example, include a product having 150 μg Nano-Colloidal Detoxified Iodine instead of potassium iodide.

In a further embodiment, iodine may be present in the nutritional composition in any one or a combination of forms disclosed herein an amount of at least about 1 mg, at least about 75 μg, at least about 80 μg, at least about 85 μg, at least about 90 μg, at least about 95 μg, at least about 100 μg, at least about 105 μg, at least about 110 μg, at least about 115 μg, at least about 120 μg, at least about 125 μg, at least about 130 μg, at least about 135 μg, at least about 140 μg, at least about 145 μg, at least about 150 μg, at least about 155 μg, at least about 160 μg, at least about 165 μg, at least about 170 μg, at least about 175 μg, at least about 180 μg, at least about 185 μg, at least about 190 μg, at least about 195 μg, at least about 200 μg, at least about 205 μg, at least about 210 μg, at least about 215 μg, at least about 220 μg, at least about 225 μg, at least about 230 μg, at least about 235 μg, at least about 240 μg, at least about 245 μg, and at least about 250 μg. Iodine may be present in the nutritional composition in a range of between and including any two of the foregoing values. In embodiments where iodine is present in a combination of forms, each constituent form may be present in a relative amount of about 10%, about 20%, about 30%, about 40%, about 50%, about 60%, about 70%, about 80%, about 90% of any one of the foregoing amounts for iodine.

The compositions, kits and methods may comprise or use magnesium. Magnesium is found primarily in both bone and muscle and is important for over 300 different enzyme reactions. A primary function of magnesium is to bind to phosphate groups in adenosine triphosphate (ATP), thereby forming a complex that assists in the transfer of ATP phosphate. Magnesium also functions within cells as a membrane stabilizer. Magnesium plays roles in nucleic acid synthesis, glycolysis, transcription of DNA and RNA, amino acid activation, membrane transport, transketolase reactions, and protein synthesis. James L. L. Groff et al., ADVANCED NUTRITION AND HUMAN METABOLISM 341 (2d ed. 1996). It is also involved in the formation of cAMP, a cytosolic second messenger that plays a role in cell signaling mechanisms. Magnesium also functions both synergistically and antagonistically with calcium in neuromuscular transmission. RDA, supra at 188. Specifically, magnesium is critical for the maintenance of electrochemical potentials of nerve and muscle membranes and the neuromuscular junction transmissions, particularly important in the heart. Not surprisingly, magnesium deficiency is tied to cardiovascular disease and hypertension. Agus et al., 17 CRIT. CARE CLIN. 175-87 (2001). Indeed, oral magnesium therapy improves endothelial function in patients with coronary disease. Shechter et al., 102 CIRCULATION 2353-58 (2000).

Magnesium is available in a variety of salts and can be included in the compositions, kits and methods in either chelated or nonchelated form. In one specific embodiment, magnesium may be included in the forms of elemental magnesium, in the form of a salt, in a chelated form, in a non-chelated form, magnesium acetate, magnesium carbonate, magnesium gluconate, magnesium chloride, magnesium citrate, magnesium silicate, magnesium stearate, magnesium sulfate, magnesium oxide, and magnesium chelated to an amino acid (magnesium glycinate, magnesium aspartate).

In another specific embodiment, magnesium may be present in the amounts ranging from about 10 mg to about 30 mg. In another embodiment, magnesium may be present in the amounts ranging from about 16 mg to about 24 mg. In another embodiment, magnesium may be present in the amounts ranging from about 18 mg to about 22 mg. In another embodiment, magnesium may be present in the amount of about 20 mg.

In another specific embodiment, magnesium may be included in specific ranges or amounts for each specific form. When provided in their specific forms, the provided numerical range or amount includes the amounts of the specific form and/or compounds that are equivalent to the specific form. For example, magnesium may be in the form magnesium oxide and may be included in the amount of about 20 mg. Accordingly, in this example, “magnesium oxide in the amount of about 20 mg” would include 20 mg of magnesium oxide and/or its equivalents and would, for example, include a product having 20 mg magnesium stearate instead of magnesium oxide.

In a further embodiment, magnesium may be present in the nutritional composition in any of one or a combination of forms disclosed herein in an amount of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, and at least about 100 mg. Magnesium may be present in the nutritional composition in a range of between and including any two of the foregoing values. In embodiments where magnesium is present in a combination of forms, each constituent form may be present in a relative amount of about 10%, about 20%, about 30%, about 40%, about 50%, about 60%, about 70%, about 80%, about 90% of any one of the foregoing amounts for magnesium.

The compositions, kits and methods may comprise or use zinc. Zinc plays a role in numerous metabolic activities such as nucleic acid production, protein synthesis, and development of the immune system. There are more than 200 zinc metalloenzymes including aldolase, alcohol dehydrogenase, RNA polymerase, and protein kinase C. Zima et al., 17 BLOOD PURIF. 182-86 (1999). Zinc stabilizes RNA and DNA structures, forms zinc fingers in nuclear receptors, and is a component of chromatin proteins involved in transcription and replication. Deficiencies of zinc during pregnancy have been shown to contribute to severe fetal abnormalities. Srinivas et al., 68(6) INDIAN J. PEDIATR. 519-22 (2001); Yang et al., 13(4) BIOMED. ENVIRON. SCI. 280-86 (2000); King, 71(Supp.) AM. J. CLIN. NUTR. 1334S-43S (2000). Indeed, the recommended daily allowance for zinc increases during pregnancy. A higher dose of zinc, however, is associated with causing nausea in some patients. Thus, for pregnant women or other patients that are more susceptible to nausea, a conservative amount of zinc that still provides adequate nutritional supplementation is desirable. Zinc is available in many forms and may be included in the compositions, kits and methods in chelated or nonchelated form.

In a specific embodiment, zinc may be provided in one or more of the forms of elemental zinc, in the form of a salt, in a chelated form, in a non-chelated form, zinc acetate, zinc gluconate, zinc picolinate, zinc sulfate and zinc oxide. In a specific embodiment, zinc may be included in the form of zinc oxide. In another specific embodiment, zinc may be included in amounts ranging from about 12.5 mg to about 37.5 mg. In another specific embodiment, zinc may be included in amounts ranging from about 20 mg to about 30 mg. In another specific embodiment, zinc may be included in amounts ranging from about 22.5 mg to about 27.5 mg. In another embodiment, zinc may be included in an amount of about 25 mg.

In another specific embodiment, zinc may be included in specific ranges or amounts for each specific form. When provided in their specific forms, the provided numerical range or amount includes the amounts of the specific form and/or compounds that are equivalent to the specific form. For example, zinc may be in the form zinc oxide and may be included in the amount of about 25 mg. Accordingly, in this example, “zinc oxide in the amount of about 25 mg” would include 25 mg of zinc oxide and/or its equivalents and would, for example, include a product having 25 mg zinc sulfate instead of zinc oxide.

In a further embodiment, zinc may be present in the nutritional composition in any of one or a combination of forms disclosed herein in an amount of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, and at least about 100 mg. Zinc may be present in the nutritional composition in a range of between and including any two of the foregoing values. In embodiments where zinc is present in a combination of forms, each constituent form may be present in a relative amount of about 10%, about 20%, about 30%, about 40%, about 50%, about 60%, about 70%, about 80%, about 90% of any one of the foregoing amounts for zinc.

The compositions, kits and methods may comprise or use copper. Copper is an important component of the process of gene expression. Additionally, one of copper's most vital roles is to help form hemoglobin, which, as previously discussed, carries oxygen to tissues via its iron component. In this respect copper plays a key role in protecting against anemia. Further, deficiencies of copper may lead to neutropenia and bone abnormalities in pregnant and lactating women. Uauy et al., AMER J CLIN NUTR 67:952S-959S (Supp.) (1998). In addition, a fetus must accumulate copper at a rate of 50 mcg×kg−1×d−1 over the latter half of pregnancy; any deficiency in accumulation may lead to low birth weight and protein-energy malnutrition. Id. Many forms of copper are known to those skilled in the art, including copper oxide (Reade Advanced Materials, Providence, R.I.). In a specific embodiment, copper may be included in the forms of a salt, in a chelated form, in a non-chelated form, cupric oxide, copper sulfate, copper gluconate, copper citrate, cupric acetate, alkaline copper carbonate, and copper salicylate.

In another specific embodiment, copper may be included in amounts ranging from about 1.0 mg to about 3.0 mg. In another specific embodiment, copper may be included in amounts ranging from about 1.6 mg to about 2.4 mg. In another specific embodiment, copper may be included in amounts ranging from about 1.8 mg to about 2.2 mg. In another embodiment, copper may be included in an amount of about 2.0 mg.

In another specific embodiment, copper may be included in specific ranges or amounts for each specific form. When provided in their specific forms, the provided numerical range or amount includes the amounts of the specific form and/or compounds that are equivalent to the specific form. For example, copper may be in the form copper oxide and may be included in the amount of about 2.0 mg. Accordingly, in this example, “copper oxide in the amount of about 2.0 mg” would include 2.0 mg of copper oxide and/or its equivalents and would, for example, include a product having 2.0 mg copper sulfate instead of copper oxide.

In a further embodiment, copper may be present in the nutritional composition in any of one or a combination of forms disclosed herein in an amount of at least about 0.5 mg, at least about 0.6 mg, at least about 0.7 mg, at least about 0.8 mg, at least about 0.9 mg, at least about 1 mg, at least about 1.1 mg, at least about 1.2 mg, at least about 1.3 mg, at least about 1.4 mg, at least about 1.5 mg, at least about 1.6 mg, at least about 1.7 mg, at least about 1.8 mg, at least about 1.9 mg, at least about 2 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0, at least about 3.1 mg, at least about 3.2 mg, at least about 3.3 mg, at least about 3.4 mg, and at least about 3.5 mg. Copper may be present in the nutritional composition in a range of between and including any two of the foregoing values. In embodiments where copper is present in a combination of forms, each constituent form may be present in a relative amount of about 10%, about 20%, about 30%, about 40%, about 50%, about 60%, about 70%, about 80%, about 90% of any one of the foregoing amounts for copper.

The compositions, kits and methods may comprise or use omega-3 fatty acids. Omega-3 fatty acids play integral roles in physiological mechanisms that serve to prevent, treat and/or alleviate the occurrence or negative effects of some diseases and has shown multiple health-promoting properties in adults. For example, omega-3 fatty acids are linked to health benefits such as preventing the occurrence of cancer, preventing the occurrence of heart disease, and are helpful in brain health and immune function. Indeed, omega-3 fatty acids include essential fatty acids linked to numerous health benefits, such as docahexaenoic acid (or docosahexaenoic acid, DHA), eicosapentaenoic acid (EPA) and a-linolenic acid (ALA). In another specific embodiment, the compositions, kits and methods may comprise or use Docahexaenoic acid (or docosahexaenoic acid, DHA). In another specific embodiment, the compositions, kits and methods may comprise or use eicosapentaenoic acid (EPA). In another specific embodiment, the compositions, kits and methods may comprise or use a-linolenic acid (ALA).

The omega-3 fatty acid DHA, a major component of fish oil, has been shown to be of particular importance, especially during pregnancy or for lowering blood pressure. Indeed, studies suggest that DHA, but not EPA, reduce ambulatory blood pressure and heart rate in hyperlipidemic men. TA Mori et al., HYPERTENSION. 34:253-260 (1999). The results of this study thus suggest that DHA is the principal fatty acid in fish and fish oils that is responsible for blood pressure and heart rate effects in humans. Id.

Further, DHA is vital for optimal fetal and infant brain/cognitive development, as well as for normal brain function throughout life. F M Rioux, O. Hernell et al., ACTA PAEDIATR 95(2):137-144 (2006). The sleep patterns of infants born to mothers with higher plasma phospholipid DHA suggest greater central nerve system maturity. S R Cheruku, C J Lammi-Keefe et al., AM J CLIN NUTR 76:608-613, 2002. Additionally, children with Attention Deficit Hyperactivity Disorder (ADHD) have been shown to have abnormal levels of DHA. EA Mitchell, M. Manku et al., CLIN PEDIATR 26:406-411 (1986); L J Stevens, J R Burgess et al., PHYSIOL BEHAV 59:915-920 (1996). Studies have indicated a correlation between maternal DHA intake and intelligence quotient in the child. The direct correlation between brain development and systemic DHA status is secondary to the fact that DHA is taken up by the brain in preference to other fatty acids. Adequate DHA levels in pregnancy have also been correlated with optimizing the length of gestation and decreasing the risk of neurodevelopmental psychopathology. These critical findings have prompted the National Institute of Health (NIH) to recommend that pregnant women consume at least 300 mg of omega-3 fatty acids during pregnancy. N. Neurenger et al., NUTR REV 44:285-294 (1986); G. Hornstra et al., AM J CLIN NUTR 71:285S-291S (2000); I B Helland et al., PEDIATRICS 111:E39-E44 (2003); F. Facchinetti et al., EUR REV MED PHARMACOL SCI 9(1):41-48 (2005); R K McNamara et al., PROSTAGLANDINS LEUKOT ESSENT FATTY ACIDS (29 Aug. 2006).

DHA is also important for the development of the infant retina and improving the visual acuity of the infant. C A Francois, W E Connor et al., AM J CLIN NUTR 77:226-233 (2003). Preterm infants have a more rapid development of visual acuity if fed human milk or formula enriched with DHA, compared to standard formula. M H Jorgensen, K F Michaelsen et al., LIPIDS 31(1):99-105 (1996). An increase in visual acuity has also been observed to develop more rapidly in term infants breast-fed from mothers whose diets are supplemented with DHA. Id.

In addition to the aforementioned benefit of DHA to the developing child, this essential fatty acid has also shown multiple health-promoting properties in adults. These include anti-thrombotic, anti-inflammatory and anti-atherosclerotic activity, all of which reduce the risk of heart disease. M Laidlaw and B J Holub, AM J CLIN NUTR 77:37-42 (2003). Inverse relationships have also been found between systemic levels of omega-3 fatty acids such as DHA and incidence and severity of mood disorders and depression, including postpartum depression. Therefore, introduction of omega-3 during pregnancy has a double benefit, to both child and mother. F B Hu et al., JAMA 287(14):1815-1821 (2002); C. Von Schacky et al., ANN INTERN MED 130:554-562 (1999); G. Parker et al., AM J PSYCHIATRY 163(6):969-978 (2006); S J Otto et al., PROSTAGLANDINS LEUKOT EsSENT FATTY ACIDS 69(3):237-243 (2003).

For women, DHA is particularly useful in counteracting the progression of breast cancer. Human breast cancer cells exposed to DHA exhibit an increase in cell death by apoptosis. B A Stoll, BR J NUTR 87(3):193-198, 2002. DHA also inhibits cyclooxygenase-2, which promotes mammary carcinogenesis. Id. DHA supplementation during pregnancy has also been shown to increase the length of gestation by about six days, helping mothers carry to a healthy full term. C M Smuts et al., OBSTETRICS AND GYNECOLOGY 101(3):469-479 (2003).

Intake of omega-3 fatty acids such as DHA not only leads to their incorporation into cell membrane lipids (B A Stoll, BR J NUTR 87(3):193-198 (2002)), but also storage in adipose tissue and secretion in breast milk. C A Francois, W E Connor et al., AM J CLIN NUTR 77:226-233 (2003). Although the human body can derive a limited amount of DHA from another fatty acid known as alpha-linolenic acid, this process is inefficient for optimal needs. A rich dietary source of direct DHA is fish. Id. However, some lactating women are vegetarians, have limited access to fish or simply do not like fish. A further problem with encouraging increased fish intake in pregnancy is that most species contain methyl mercury (MeHg) in various amounts. MeHg is a potent neurotoxin that can increase the risk of retarded cognitive development. This concern prompted both the United States Environmental Protection Agency (2004) and the Food and Drug Administration (2001) to issue advisories recommending that pregnant women modify their fish consumption. These recommendations have resulted in a reduced intake of fish during pregnancy, thus helping to protect against fetal MeHg related harm. However, this has concurrently reduced maternal intake of DHA. In fact, a recent dietary study of over 100 pregnant or nursing women in the United States showed an astonishingly low intake of DHA on average (60-80 mg/day), and a dangerously low percentage (<2) consumed the aforementioned recommended intake of 300 mg/day of DHA as set forth by the NIH. J T Cohen et al., AM J PREV MED, 29:353-365 (2005); U.S. Department of Health and Human Services, U.S. Environmental Protection Agency, “What you need to know about mercury in fish and shellfish,” Report EPA-823-F-04-009 (March 2004); E. Oken et al., OBSTET GYNECOL 102:346-351 (2003).

DHA may be obtained in solid form, such as in a whole-cell microbial product, or in liquid form, such as in an oil. An example of DHA in oil form is DHASCO®-T vegetable oil from micro-algae (Martek Biosciences Corporation, Columbia, Md.). In a specific composition, the DHA is DHAgold®, life's DHA™ (DHASCO®), any Algae Oil, Krill Oil and/or vegetarian DHA.

In a specific embodiment, the source of DHA may be from one or more of animal, fish, plants, algae or microorganism production.

In another embodiment, the compositions, kits and methods may include DHA derived from algae. DHA derived from algae, as opposed to being derived from fish oil, has numerous beneficial effects. First, the DHA from algae does not have the “fishy” smell that can come with DHA from fish oil. Indeed, high doses of DHA from fish oil may result in the patient having an unappealing after taste or a slight “fishy” body odor or “fishy” odor on the patient's breath. Second, DHA derived from algae can be more easily regulated to assure consistency and further remove the risk of added chemicals or other dangers. For example, DHA from algae would not have the risk of being tainted with mercury as opposed to DHA from fish oil. Thus, DHA from algae provides pregnant women and neonate with DHA without this risk and dangers of mercury. In a specific embodiment, the source of DHA may be from algae oil. In another specific embodiment, the source of algae oil may be one or more of microalgae Schizochytrium sp, microalgae Crypthecodinium cohnii, microalgae Ulkenia sp. SAM2179, microalgae Schizochytrium linacinum strain SC-1. In another specific embodiment the source of DHA may be Martek Oil C53-O100.

In another specific embodiment, omega-3 fatty acids may be included in amounts ranging from about 100 mg to about 300 mg. In another specific embodiment, omega-3 fatty acids may be included in amounts ranging from about 160 mg to about 240 mg. In another specific embodiment, omega-3 fatty acids may be included in amounts ranging from about 180 mg to about 220 mg. In another embodiment, omega-3 fatty acids may be included in an amount of about 200 mg.

In another specific embodiment, omega-3 fatty acids may be included in specific ranges or amounts for each specific form. When provided in their specific forms, the provided numerical range or amount includes the amounts of the specific form and/or compounds that are equivalent to the specific form. For example, omega-3 fatty acids may be in the form of DHA and may be included in the amount of about 200 mg. Accordingly, in this example, “DHA in the amount of about 200 mg” would include 200 mg of DHA and/or its equivalents and would, for example, include a product having 200 mg EPA instead of DHA.

In another specific embodiment, omega-3 fatty acids may be in the form of DHA and may be included in amounts ranging from about 100 mg to about 300 mg. In another specific embodiment, omega-3 fatty acids in the form of DHA may be included in amounts ranging from about 160 mg to about 240 mg. In another specific embodiment, omega-3 fatty acids in the form of DHA may be included in amounts ranging from about 180 mg to about 220 mg. In another embodiment, omega-3 fatty acids in the form of DHA may be included in an amount of about 200 mg.

In another specific embodiment, the source of omega-3 fatty acids may be algal oil. The algal oil may be one or more of algae oil may be one or more of microalgae Schizochytrium sp, microalgae Crypthecodinium cohnii, microalgae Ulkenia sp. SAM2179, microalgae Schizochytrium linacinum strain SC-1. In another specific embodiment the source of DHA may be Martek Oil C53-O100. The algal oil may be present in the nutritional composition in any one or a combination of forms disclosed herein in an amount of at least about 200 mg, at least about 210 mg, at least about 215 mg, at least about 220 mg, at least about 225 mg, at least about 230 mg, at least about 235 mg, at least about 240 mg, at least about 245 mg, at least about 250 mg, at least about 255 mg, at least about 260 mg, at least about 265 mg, at least about 270 mg, at least about 275 mg, at least about 280 mg, at least about 285 mg, at least about 290 mg, at least about 295 mg, at least about 230 mg, at least about 300 mg, at least about 305 mg, at least about 310 mg, at least about 315 mg, at least about 320 mg, at least about 325 mg, at least about 330 mg, at least about 335 mg, at least about 340 mg, at least about 345 mg, at least about 350 mg, at least about 355 mg, at least about 360 mg, at least about 365 mg, at least about 370 mg, at least about 375 mg, at least about 380 mg, at least about 385 mg, at least about 390 mg, at least about 395 mg, at least about 400 mg, at least about 405 mg, at least about 410 mg, at least about 415 mg, at least about 420 mg, at least about 425, at least about 430 mg, at least about 435 mg, at least about 440 mg, at least about 445 mg, and at least about 450 mg. The source of omega-3 fatty acids may be present in the nutritional composition in a range of between and including any two of the foregoing values. In embodiments where the source of omega-3 fatty acids is present in a combination of forms, each constituent form may be present in a relative amount of about 10%, about 20%, about 30%, about 40%, about 50%, about 60%, about 70%, about 80%, about 90% of any one of the foregoing amounts.

It is understood that for the amounts provided for the source of omega-3 fatty acids, the content of the omega-3 fatty acid, such as DHA, may be in an amount less than the total. For example, the source of omega-3 fatty acid may be present in an amount of 415 mg and may comprise DHA in an amount of about 200 mg.

The compositions, kits and methods may include or use a combination of the included vitamins, nutrients and minerals just described. In a specific embodiment, the compositions, kits and methods may include vitamin D, iodine, vitamin B1, vitamin B6, vitamin B12, vitamin B2, vitamin B9, vitamin B3, vitamin E, vitamin A, vitamin C, iron, zinc, copper, magnesium, omega 3 fatty acids and one or more pharmaceutically acceptable carriers.

In another embodiment, the vitamins, nutrients and minerals may be included or used in any specific form just described. In a specific embodiment, the omega 3 fatty acids may be DHA.

In another embodiment, the compositions, kits and methods may include or use a combination of the included vitamins, nutrients and minerals in the ranges or amounts just described.

In a specific embodiment, the compositions, kits and methods may include or use vitamin D in an amount of about 500 I.U. to about 1500 I.U., iodine in an amount of about 75 μg to about 225 μg, vitamin B1 in an amount of about 0.8 mg to about 2.4 mg, vitamin B6 in an amount of about 1.2 mg to about 3.8 mg, vitamin B12 in an amount of about 6 μg to about 18 μg, vitamin B2 in an amount of about 0.9 mg to about 2.7 mg, vitamin B9 in an amount of about 0.5 mg to about 1.5 mg, vitamin E in an amount of about 10 I.U. to about 30 I.U., vitamin A in an amount of about 550 I.U. to about 1650 I.U., vitamin C in an amount of about 15 mg to about 45 mg, vitamin B3 in an amount of about 7.5 mg to about 22.5 mg, iron in an amount of about 14.5 mg to about 43.5 mg, zinc in an amount of about 12.5 mg to about 37.5 mg, copper in an amount of about 1.0 mg to about 3.0 mg, magnesium in an amount of about 10 mg to about 30 mg, and omega 3 fatty acids comprising DHA in an amount of about 100 mg to about 300 mg. In a specific embodiment, the compositions, kits and methods described herein may include or use vitamin D in an amount of about 1000 I.U., iodine in an amount of about 150 μg, vitamin B1 in an amount of about 1.6 mg, vitamin B6 in an amount of about 2.5 mg, vitamin B12 in an amount of about 12 μg, vitamin B2 in an amount of about 1.8 mg, vitamin B9 in an amount of about 1.0 mg, vitamin E in an amount of about 20 I.U., vitamin A in an amount of about 1100 I.U., vitamin C in an amount of about 30 mg, vitamin B3 in an amount of about 15 mg, iron in an amount of about 29 mg, zinc in an amount of about 25 mg, copper in an amount of about 2.0 mg, magnesium in an amount of about 20 mg, and omega 3 fatty acids comprising DHA in an amount of about 200 mg.

In one embodiment, a nutritional composition comprises at least about 1100 IU vitamin A, at least about 60 mg vitamin C, at least about 1000 IU vitamin D, at least about 20 IU vitamin E, at least about 1.6 mg vitamin B1, at least about 1.8 mg B2, at least about 15 mg B3, at least about 1 mg vitamin B9, at least about 25 μg vitamin B12, at least about 90 mg iron, at least about 150 μg iodine, at least about 20 mg magnesium, at least about 25 mg zinc, at least about 2 mg copper and at least about 400 mg of the source of omega-3 fatty acids, which may comprise at least about 200 mg of DHA. The nutritional composition may be encapsulated by a hard shell or soft shell capsule or may be coated by a polymeric, enteric or sugar coating. In embodiments where the nutritional composition is encapsulated by a soft shell capsule, the soft shell capsule may be formulated from a gelatin or non-gelatin composition that does not comprise ingredients from any animal sources.

In one embodiment, vitamin D3, iodine, vitamin B1, vitamin B6, vitamin B12, vitamin B2, vitamin B9, vitamin B3, vitamin E, vitamin A, vitamin C, iron, zinc, copper, magnesium, and omega 3 fatty acids may be provided in one composition. In a specific embodiment, the composition may be in the dosage form of a soft shell capsule, such as a gel-cap. To ensure that all such ingredients may be provided in one soft shell capsule composition, various inactive ingredients and pharmaceutically acceptable carries may be added. In a specific embodiment, a wetting, stabilizing agent may be used for the soft shell capsule composition. In a specific embodiment, the wetting or stabilizing agent is lecithin oil. In another specific embodiment beeswax may be added. In another specific embodiment, soybean oil may be added.

In another embodiment, invention kit comprising one or more compositions may be provided. In a specific embodiment, the one or more compositions may include or use a combination of the vitamins, nutrients and minerals just described. In a specific embodiment, the one or more compositions may collectively use or include vitamin D, iodine, vitamin B1, vitamin B6, vitamin B12, vitamin B2, vitamin B9, vitamin B3, vitamin E, vitamin A, vitamin C, iron, zinc, copper, magnesium, omega 3 fatty acids and one or more pharmaceutically acceptable carriers.

In a specific embodiment, the two compositions may collectively use or include vitamin D, iodine, vitamin B1, vitamin B6, vitamin B12, vitamin B2, vitamin B9, vitamin B3, vitamin E, vitamin A, vitamin C, iron, zinc, copper, magnesium, omega 3 fatty acids and one or more pharmaceutically acceptable carriers.

In a specific embodiment, the multiple compositions, kits and methods may collectively include or use vitamin D in an amount of about 500 I.U. to about 1500 I.U., iodine in an amount of about 75 μg to about 225 μg, vitamin B1 in an amount of about 0.8 mg to about 2.4 mg, vitamin B6 in an amount of about 1.2 mg to about 3.8 mg, vitamin B12 in an amount of about 6 μg to about 18 μg, vitamin B2 in an amount of about 0.9 mg to about 2.7 mg, vitamin B9 in an amount of about 0.5 mg to about 1.5 mg, vitamin E in an amount of about 10 I.U. to about 30 I.U., vitamin A in an amount of about 550 I.U. to about 1650 I.U., vitamin C in an amount of about 15 mg to about 45 mg, vitamin B3 in an amount of about 7.5 mg to about 22.5 mg, iron in an amount of about 14.5 mg to about 43.5 mg, zinc in an amount of about 12.5 mg to about 37.5 mg, copper in an amount of about 1.0 mg to about 3.0 mg, magnesium in an amount of about 10 mg to about 30 mg, and omega 3 fatty acids comprising DHA in an amount of about 100 mg to about 300 mg.

In a specific embodiment, the multiple compositions, kits and methods may collectively include or use vitamin D in an amount of about 1000 I.U., iodine in an amount of about 150 μg, vitamin B1 in an amount of about 1.6 mg, vitamin B6 in an amount of about 2.5 mg, vitamin B12 in an amount of about 12 μg, vitamin B2 in an amount of about 1.8 mg, vitamin B9 in an amount of about 1.0 mg, vitamin E in an amount of about 20 I.U., vitamin A in an amount of about 1100 I.U., vitamin C in an amount of about 30 mg, vitamin B3 in an amount of about 15 mg, iron in an amount of about 29 mg, zinc in an amount of about 25 mg, copper in an amount of about 2.0 mg, magnesium in an amount of about 20 mg, and omega 3 fatty acids comprising DHA in an amount of about 200 mg

In one embodiment, a kit comprising a nutritional composition and an adjuvant composition may be provided. The nutritional composition may comprise vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids, and one or more pharmaceutically-acceptable carriers. The adjuvant composition may be formulated to mitigate at least one undesired side effect associated with the administering of the nutritional composition to a patient.

In one embodiment, the nutritional composition may comprise relatively high levels of iron of at least about 50 mg, at least about 60 mg, at least about 70 mg, at least about 80 mg, and at least about 90 mg. In such embodiments, one undesired side effect associated with the administering of the nutritional composition is constipation and possibly other gastric side effects. Accordingly, in such embodiments, the adjuvant composition may comprise fiber and/or a laxative to mitigate possible constipation that may be experienced from the administering of the nutritional composition.

The fiber may be any dietary fiber known in the art. The fiber may be provided in any number of forms, including capsules, powders, and tablets. The fiber may be extracted natural fiber that include lignin, cellulose, pectin, gum, and psyllium. The fiber may also be provided in combination with polydextrose, polyols, and/or maltodextrins.

Laxatives may include those known in the art. Laxatives contain chemicals that help increase stool motility, bulk and frequency, thus relieving constipation. The laxatives may come in the form of pills, capsules, tablets, caplets, soft shell capsules such as gel-caps, liquids, suppositories, and enemas. The laxative may be a lubricant laxative which makes stools slippery. Lubricant laxatives may comprise mineral oil or other oil which can add a slick layer to the intestine's walls and stops the stool from drying out. The laxative may be an emollient laxative or stool softener. Examples of such emollient laxatives that comprises a surfactant. One specific laxative embodiment is docusate sodium, a laxative that helps to “wet” and soften the stool. The laxatives may be an osmotic or hyperosmolar laxative, which draw fluids into the intestine from the surrounding tissue. The laxative may be a stimulant laxative which works by stimulating the lining of the intestine, thereby accelerating the stool's journey through the colon. Stimulant laxative also increase a stool's hydration.

The adjuvant composition may comprise one or more sources of dietary fiber, one or more laxatives, or a combination of one or more dietary fiber and laxatives.

The adjuvant composition may be included in the composition comprising the vitamins, minerals, DHA and optionally other nutrients to provide a single dosage form that includes both the nutritional composition and the adjuvant composition. Alternatively, the adjuvant composition may be provided as a dosage form that is separate from the composition comprising the vitamins, minerals, DHA and optionally other nutrients. In this alternate embodiment, the two dosage forms may be blister packaged together and the packaging or labeling may indicate that co-administration of the adjuvant composition is optional.

The dosage forms that include the adjuvant composition may be provided in any of the usual media. For liquid preparations (e.g., suspensions, elixirs, and solutions), media containing, for example water, oils, alcohols, flavoring agents, preservatives, coloring agents and the like may be used. Pharmaceutical acceptable carriers such as starches, sugars, diluents, granulating agents, lubricants, binders, disintegrating agents and the like may be used to prepare oral solids (e.g., powders, caplets, pills, tablets, capsules, and lozenges). Controlled release forms may also be used. Because of their ease in administration, liquid or soft shell capsules, such as gel-caps, caplets, tablets, pills, and capsules represent the most advantageous oral dosage unit form. If desired, tablets may be sugar coated or enteric coated by standard techniques. All of these pharmaceutical carriers and formulations are well known to those of ordinary skill in the art. See, e.g., WADE & WALLER, HANDBOOK OF PHARMACEUTICAL EXCIPIENTS (2nd ed. 1994).

The adjuvant composition may therefore comprise fiber and/or a laxative such as docusate sodium and one or more pharmaceutically-acceptable carriers in a suitable dosage form, preferably a soft shell capsule such as a gel-cap. The docusate sodium may be provided in an amount of at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, and at least about 100 mg. The docusate may be present in the nutritional composition in a range of between and including any two of the foregoing values.

In a specific embodiment, active ingredients such as the vitamins, minerals, laxatives and nutrients, may be included in overages. Adding overages of these compounds may be necessary to meet the amounts claimed on the product label and product insert to ensure that those recited amounts are met throughout the shelf life of the product. Indeed, because of US regulatory requirements that label values reflect minimum contents of these nutrients, deviations in actual nutrient content from label values are usually thought to tend toward overages. Dwyer et al., ANAL BIOANAL CHEM, 389:37-46 (2007). In a specific embodiment, one or more of the vitamins, minerals and nutrients may be included in the compositions and methods in overages of the recited, specific label amounts of about 100% to about 150% of the label amount, although the overages are dependent on the stability of each ingredient. For example, overages of vitamin D and vitamin B12 may be necessary due to the lack of stability of specific forms. In another example, 5-methyltetrahydrofolate, a form of vitamin B9, is degraded by light, temperature and may degrade during processing and storage. Overages may be larger for some vitamins—particularly those that are less stable and more likely to deteriorate with a long shelf life, those that have other functions (such as antioxidants) in the product itself; for minerals, excess amounts with large overages are probably less likely because of their increased bulk and shelf life stability. Dwyer et al., ANAL BIOANAL CHEM, 389:37-46 (2007). Accordingly, when overages are included for any specific active ingredient, at some point in time, these ingredients with overages will degrade so that they fall within the amounts provided in the specific label. Thus, there is no literal difference between the amounts for active ingredients that include overages, and those amounts listed on the specific label. Furthermore, overages provide an equivalent efficacy of the active ingredient over the shelf life of the product. Accordingly, an active ingredient provided in overage amounts is an insubstantial change and performs substantially the same function, in substantially the same way, and leads to substantially the same result as that same active ingredient in the amounts as provided on the specific label.

In another embodiment, the nutritional supplements may include multiple vitamins, nutrients and minerals in one composition. Providing a single composition multivitamin and multinutrient supplement is an appealing feature because it improves patient compliance. Patients, and specifically for example, pregnant patients, often have nausea, and may have difficulties taking multiple pills. A one pill or one composition nutritional supplement that includes the beneficial vitamins, nutrients and minerals in appropriate dosage amounts would thus be beneficial for improving patient compliance in for example, pregnant women. In a specific embodiment one or more of the compositions may be in the dosage form of a gel-cap.

In another specific embodiment, the compositions, kits and methods of the present invention may be in the form of a liquid gel-cap which may consist of a filler comprising one or more pharmaceutically active materials dissolved or dispersed in an appropriate liquid vehicle encapsulated in a gelatin shell generally comprising gelatin together with a plasticizer such as glycerin or sorbitol.

A liquid soft shell capsule, such as a gel-cap has numerous advantages. First, it retains many of the advantages of consumer acceptance and is easier to swallow due to the outer coating being a soft and elastic gelatin shell. Also, liquid compositions are well suited for encapsulation within a soft gelatin shell, creating flexibility that further assists in the capsule being easier to swallow. The active drug contained in the liquid form also provides advantages by dispersing the drug to the active site. For example, the active drug does not first have to dissolve in the gastrointestinal tract, thereby facilitating absorption of the pharmacologically active substance. See, for example, U.S. Pat. No. 6,689,382, which is expressly incorporated by reference herein. Other formulations take advantage of the liquid form by creating a sustained release gelatin capsule, thereby permitting the delivery of the drug in a controlled fashion. See, for example, U.S. Pat. Nos. 5,324,280 and 6,929,803, which are expressly incorporated by reference herein. The filler material may comprise, for example, polyethylene glycols. See, for example, U.S. Pat. Nos. 4,780,316; 5,419,916; 5,641,512; and 6,589,536 which are expressly incorporated by reference herein. Many shell and fill formulations are discussed in “Advances in Softgel Formulation Technology”, M. S. Patel, F. S. S. Morton and H. Seager, Manufacturing Chemists, July 1989; “Soft Elastic Gelatin Capsules: A Unique Dosage Form”, William R. Ebert, Pharmaceutical Technology, October 1977; and “Soft gelatin capsules: a solution to many tableting problems”, H. Seager, Pharmaceutical Technology, September 1985.

In a specific embodiment, kits or compositions may be provided in the dosage form of a soft shell capsule. A soft-gel is a one-piece, sealed, soft gelatin shell that contains a solution, a suspension, or a semi-solid paste. Soft-gels are predominantly used to contain liquids wherein the active ingredients are present in the dissolved or suspended state. Soft-gels have been widely known and used for many years and for a variety of purposes. Because soft-gels have properties that are quite different from two-piece, hard shell capsules, the soft-gels are capable of retaining a liquid fill material. Soft-gels are often used to encapsulate consumable materials, including vitamins, dietary supplements, pharmaceuticals, and the like, in a liquid vehicle or carrier. Soft-gels arc a unique dosage form that can provide distinct advantages over more traditional dosage forms such as tablets, hard-shell capsules, and liquids. These advantages include patient compliance and consumer preference, improved bioavailability, speed of product development in many cases, shortened manufacturing time, enhanced drug stability due to less exposure of the active ingredient to oxygen, excellent dose uniformity, and product differentiation.

In another embodiment, the nutritional supplements may include multiple vitamins, nutrients and minerals in more than one composition. In a specific embodiment, various active ingredients may be incorporated into multiple compositions as a kit. In one example, fat soluble compounds such as omega 3 fatty acids, may be included in one composition, whereas water soluble vitamins such as B-complex vitamins and vitamin C may be separated into another composition. In another example, the multiple compositions may be separated due to size or the large dosage amounts of specific ingredients. In another example, the nutritional supplementation of a multivitamin may not be adequate in one composition. Accordingly, all the active ingredients may be divided into a total of two compositions, three compositions, four compositions and five composition. In one embodiment, each composition may have equal amounts of each active ingredient. In another embodiment, compositions may have unequal amounts of various active ingredients, or merely supplemental amounts of specific active ingredients.

In one preferred embodiment, the kit comprises a first composition and a second composition. The first and second compositions may be combined in a single dosage form or they may be provided as separate dosage forms. In the preferred embodiment, the first and second compositions are provided as separate dosage forms. The first composition may comprise vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids, and one or more pharmaceutically-acceptable carriers. In a preferred embodiment, the iron is present in an amount of at least about 80 mg iron, at least about 85 mg iron, at least about 90 mg iron, at least about 95 mg iron, and at least about 100 mg of iron. The iron may be present in an amount between and including any two of the foregoing values. The second composition may comprise an adjuvant composition comprising a laxative and one or more pharmaceutically-acceptable carriers. In the preferred embodiment, the laxative is a docusate sodium. The docusate sodium may be present in an amount of at least about 25 mg, at least about 30 mg, at least about 35 mg, at least about 40 mg, at least about 45 mg, at least about 50 mg, at least about 55 mg, at least about 60 mg, at least about 65, at least about 70 mg, and at least about 75 mg.

In one embodiment, the first composition may be provided in a first filler composition that is encapsulated by a first outer coating. The first filler composition may comprise any one or a combination of solid or liquid pharmaceutically-acceptable carriers, described above, and the first outer coating may be any one of a hard shell, such as a capsule shell, a soft shell, such as a gelatin, starch or vegetable-based shell, or a coating, such as a polymeric, enteric or sugar coating. In one embodiment, the first composition is vegan-friendly and both the first outer coating and the filler composition excludes gelatin and other animal-based products.

In another embodiment, the second composition may be provided in a second filler composition that is encapsulated by a second outer coating. The composition of the second filler composition may be the same as or different from the composition of the first filler composition.

The composition of the second outer coating may be the same as or different from the first outer coating. Again, the second filler composition may comprise any one or a combination of solid or liquid pharmaceutically-acceptable carriers, described above, and the second outer coating may also be any one of a hard shell, such as a capsule shell, a soft shell, such as a gelatin, starch or vegetable-based shell, or a coating, such as a polymeric, enteric or sugar coating. Again, the second composition may be provided in a vegan-friendly form in which both the second filler composition and the second outer coating excludes gelatin and other animal-based products.

The first and second outer coating may at least partially or completely encapsulate the first and second filter composition, respectively. In one embodiment, the first and second outer coating comprises gelatin.

In yet a further embodiment, the first and second composition may be provided in a single dosage form. Thus, the first and second composition may be admixed in the same filler composition and encapsulated together by an outer coating.

In another specific embodiment, the composition, kits and methods may be used as a dietary supplement. In another embodiment, the composition, kits and methods may be used as a prescription prenatal vitamin. In another embodiment, the compositions, kits and methods, may be administered to a patient, such as a woman during pregnancy, prenatal or who is breast-feeding. In another embodiment, the compositions, kits and methods may be utilized or administered, once a day, twice a day, three times a day, four times a day and five times a day. When multiple compositions are provided in a kit, the compositions may be co-administered at the same or administered separately.

The compositions, kits and methods may be used or utilized in one or more dosage forms. In a specific embodiment, the dosage form more be a capsule, tablet, caplet, a soft shell capsule, gel caplet (gel-cap), syrup, a liquid composition, a concentrated powder, and a concentrated powder admixed with a liquid. The kits may comprise multiple compositions utilizing multiple dosage forms.

The ingredients of the compositions disclosed herein may thus be combined into a capsule, tablet, caplet, soft shell capsule, gel caplet (gel-cap), syrup, a liquid composition, a concentrated powder, and a concentrated powder admixed with a liquid, and which may be administered alone or in suitable combination with other components. For example, the composition may be administered in one or more caplets or gel caps as practical for ease of administration. Each of the vitamins, nutrients and minerals is commercially available, and can be blended to form a single composition or can form multiple compositions, which may be co-administered. In a specific embodiment one or more of the compositions may be in the dosage form of a soft shell capsule or a gel-cap.

To prepare the compositions, each of the active ingredients may be combined in intimate admixture with a suitable carrier according to conventional compounding techniques. The carrier may take a wide variety of forms depending upon the form of the preparation desired for administration, e.g., oral, sublingual, nasal, topical patch, or parenteral.

In preparing the composition in oral dosage form, any of the usual media may be utilized. For liquid preparations (e.g., suspensions, elixirs, and solutions), media containing, for example water, oils, alcohols, flavoring agents, preservatives, coloring agents and the like may be used. Pharmaceutical acceptable carriers such as starches, sugars, diluents, granulating agents, lubricants, binders, disintegrating agents and the like may be used to prepare oral solids (e.g., powders, caplets, pills, tablets, capsules, and lozenges). Controlled release forms may also be used. Because of their ease in administration, caplets, tablets, pills, and capsules represent the most advantageous oral dosage unit form, in which case solid carriers are employed. If desired, tablets may be sugar coated or enteric coated by standard techniques. All of these pharmaceutical carriers and formulations are well known to those of ordinary skill in the art. See, e.g., WADE & WALLER, HANDBOOK OF PHARMACEUTICAL EXCIPIENTS (2nd ed. 1994).

In a specific mode of administration, the dosage forms, may be swallowable, chewable or dissolvable.

Swallowable compositions are well known in the art and are those that do not readily dissolve when placed in the mouth and may be swallowed whole without any chewing or discomfort. In a specific embodiment, the swallowable compositions may have a shape containing no sharp edges and a smooth, uniform and substantially bubble free outer coating.

To prepare the swallowable compositions, each of the active ingredients may be combined in intimate admixture with a suitable carrier according to conventional compounding techniques. In a specific embodiment of the swallowable compositions, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions from atmospheric degradation. Polymeric films that may be used in preparing the swallowable compositions include vinyl polymers such as polyvinylpyrrolidone, polyvinyl alcohol and acetate, cellulosic such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia Pharmaceutical carriers and formulations for swallowable compounds are well known to those of ordinary skill in the art. See generally, e.g., WADE & WALLER, HANDBOOK OF PHARMACEUTICAL EXCIPIENTS (2nd ed. 1994).

Chewable compositions are those that have a palatable taste and mouthfeel, are relatively soft and quickly break into smaller pieces and begin to dissolve after chewing such that they are swallowed substantially as a solution.

In order to create chewable compositions, certain ingredients should be included to achieve the attributes just described. For example, chewable compositions should include ingredients that create pleasant flavor and mouthfeel and promote relative softness and dissolvability in the mouth. The following discussion describes ingredients that may help to achieve these characteristics.

Chewable compositions preferably have a pleasant or palatable flavor and a pleasant mouthfeel. A variety of ingredients can be included in the compositions enhance mouthfeel.

In the chewable compositions, sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Further, by way of example and without limitation, fondant or gums such as gelatin, agar, arabic gum, guar gum, and carrageenan may be added to improve the chewiness of the compositions. Fatty materials that may be included include, by way of example and without limitation, vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of the chewable nutritional supplement compositions described herein. By “enhance the texture” it is meant that the alkyl polysiloxane improves one or more of the stiffness, the brittleness, and the chewiness of the chewable supplement, relative to the same preparation lacking the alkyl polysiloxane. By “enhance the mouthfeel” it is meant that the alkyl polysiloxane reduces the gritty texture of the supplement once it has liquefied in the mouth, relative to the same preparation lacking the alkyl polysiloxane.

Alkyl polysiloxanes generally comprise a silicon and oxygen-containing polymeric backbone with one or more alkyl groups pending from the silicon atoms of the back bone. Depending upon their grade, they can further comprise silica gel. Alkyl polysiloxanes are generally viscous oils. Exemplary alkyl polysiloxanes that can be used in the swallowable, chewable or dissolvable compositions include, by way of example and without limitation, monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation, which contains 30% simethicone USP. Simethicone USP contains not less than about 90.5% by weight (CH₃)₃—Si{OSi(CH₃)₂}CH₃ in admixture with about 4.0% to about 7.0% by weight SiO₂.

Chewable compositions should begin to break and dissolve in the mouth shortly after chewing begins such that the compositions can be swallowed substantially as a solution. The dissolution profile of chewable compositions may be enhanced by including rapidly water-soluble fillers and excipients. Rapidly water-soluble fillers and excipients preferably dissolve within about 60 seconds of being wetted with saliva. Indeed, it is contemplated that if enough water-soluble excipients are included in the compositions, they may become dissolvable rather than chewable composition forms. Examples of rapidly water soluble fillers suitable for use include, by way of example and without limitation, saccharides, amino acids and the like. Disintegrants also may be included in the compositions in order to facilitate dissolution. Disintegrants, including permeabilizing and wicking agents, are capable of drawing water or saliva up into the compositions which promotes dissolution from the inside as well as the outside of the compositions. Such disintegrants, permeabilizing and/or wicking agents that may be used include, by way of example and without limitation, starches, such as corn starch, potato starch, pre-gelatinized and modified starches thereof, cellulosic agents, such as Ac-di-sol, montrnorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable, chewable and/or dissolvable compositions so long as they are consistent with the objectives described herein. For example, binders, are substances used to cause adhesion of powder particles in granulations. Such compounds appropriate for use include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol and others known to those of ordinary skill in the art.

Diluents also may be included in the compositions in order to enhance the granulation of the compositions. Diluents can include, by way of example and without limitation, microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycerin, and their mixtures.

Lubricants are substances used in composition formulations that reduce friction during composition compression. Lubricants that may be used include, by way of example and without limitation, stearic acid, calcium stearate, magnesium stearate, zinc stearate, talc, mineral and vegetable oils, benzoic acid, poly(ethylene glycol), glyceryl behenate, stearyl futmarate, and others known to those of ordinary skill in the art.

Glidants improve the flow of powder blends during manufacturing and minimize composition weight variation. Glidants that may be used include, by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents, such as grape skin extract, beet red powder, beta carotene, annato, carmine, turmeric, paprika and others known to those of ordinary skill in the art. It is recognized that no colorant is required in the nutritional supplement compositions described herein.

If desired, the compositions may be sugar coated or enteric coated by standard techniques. The unit dose forms may be individually wrapped, packaged as multiple units on paper strips or in vials of any size, without limitation. The swallowable, chewable or dissolvable compositions may be packaged in unit dose, rolls, bulk bottles, blister packs and combinations thereof, without limitation.

The swallowable, chewable or dissolvable compositions may be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable nutritional supplements with improved mouthfeel. U.S. Pat. No. 5,965,162, relates to kits and methods for preparing multivitamin comestible units which disintegrate quickly in the mouth, especially when chewed. Further, all pharmaceutical carriers and formulations described herein are well known to those of ordinary skill in the art, and determination of workable proportions in any particular instance will generally be within the capability of the person skilled in the art. Details concerning any of the excipients may be found in WADE & WALLER, HANDBOOK OF PHARMACEUTICAL EXCIPIENTS (2nd ed. 1994). AU active ingredients, fillers and excipients are commercially available from companies such as Aldrich Chemical Co., FMC Corp, Bayer, BASF, Alexi Fres, Witco, Mallinckrodt, Rhodia, ISP, and others.

A specific embodiment may comprise kits or swallowable compositions packaged in blister packs. Blister packs as packaging for swallowable compositions are well known to those of ordinary skill in the art. Blister packs may be made of a transparent plastic sheet which has been formed to carry a matrix of depression or blisters. One or more swallowable compositions are received in each depression or blister. A foil or plastic backing is then adhered across the plane of the sheet sealing the swallowable compositions in their respective blisters. Examples of materials used for the blister packs include, but are not limited to, aluminum, paper, polyester, PVC, and polypropylene. Alternative materials are known to those of ordinary skill in the art. To remove a swallowable composition, the depression material is pressed in and the composition is pushed through the backing material. Multiple blister packs may be placed in an outer package, often a box or carton for sale and distribution.

Another specific embodiment may comprise kits or swallowable compositions packaged in bottles. The bottle may be glass or plastic in form with a pop or screw top cap. Bottle packaging for compositions in swallowable form are well known to those of ordinary skill in the art.

Additionally, the unit dose forms may be individually wrapped, packaged as multiple units on paper strips or in vials of any size, without limitation. The swallowable, chewable or dissolvable compositions may be packaged in unit dose, rolls, bulk bottles, blister packs and combinations thereof, without limitation.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

Without further elaboration, it is believed that one skilled in the art, using the preceding description, can utilize the present invention to the fullest extent. The following examples are illustrative only, and not limiting of the remainder of the disclosure in any way whatsoever.

### Example 1

A kit comprising the following compositions was prepared in a soft shell capsule form, such as a gel-cap, including the appropriate excipients, by standard methods known to those of ordinary skill in the art:

Soft Shell Capsule #1

| | |
|---|---|
| Vitamin A (Beta Carotene) | 1100 IU |
| Vitamin C | 60 mg |
| Vitamin D (Vitamin D3) | 1000 IU |
| Vitamin E | 20 IU |
| Vitamin B1 | 1.6 mg |
| Vitamin B2 (Riboflavin) | 1.8 mg |
| Vitamin B3 (Niacinamide) | 15 mg |
| Vitamin B6 | 2.5 mg |
| Vitamin B9 (Folic Acid) | 1 mg |
| Vitamin B12 | 25 μg |
| Iron | 90 mg |
| Iodine | 150 μg |
| Magnesium | 20 mg |
| Zinc | 25 mg |
| Copper | 2.0 mg |
| DHA | 200 mg |

Soft Shell Capsule #1 may contain any one or more of the following optional ingredients: gelatin, soybean oil, sorbitol, glycerol, yellow beeswax, USP purified water, lecithin, titanium dioxide, FD&C Red #40, FD&C Blue #1, white edible ink, soy, corn oil, DL alpha tocopherol, and medium chain triglycerides.

Soft Shell Capsule #2

| | |
|---|---|
| Docusate sodium | 50 mg |

Soft Shell Capsule #2 may contain any one or more of the following optional ingredients: gelatin, sorbitol, polyethylene glycol, glycerin, purified water, propylene glycol, titanium dioxide, citric acid, and edible ink.

The soft shell capsules may be provided in a blister pack and arranged such that soft shell capsule #1 and #2 together constitute a single recommended dose. Instructions may be provided for the co-administration of soft shell capsule #1 and #2 at substantially the same time or at different times. Instructions may also be provided that the administration of soft shell capsule #2 is optional on an as-needed basis in the event certain undesirable side effects are being experienced, such as constipation.

### Example 2

A kit comprising the following compositions was prepared in soft shell capsule form, including the appropriate excipients, by standard methods known to those of ordinary skill in the art:

Soft Shell Capsule #1

| | |
|---|---|
| Vitamin A (Beta Carotene) | 1100 IU |
| Vitamin C (Ascorbic Acid) | 60 mg |
| Vitamin D (Cholecalciferol) | 1000 IU |
| Vitamin E (dl-alpha tocopheryl acetate) | 20 IU |
| Vitamin B1(Thiamin) | 1.6 mg |
| Vitamin B2 (Riboflavin) | 1.8 mg |
| Vitamin B3 (Niacin or Niacinamide) | 15 mg |
| Vitamin B6 (Pyridoxine Hydrochloride) | 2.5 mg |
| Vitamin B9 (Folic Acid 0.4 mg and L-Methylfolate | 1 mg |
| Calcium 0.6 mg) | |
| Vitamin B12 (Cyanocobalamin) | 25 μg |
| Iron (Polysaccharide Iron Complex) | 90 mg |
| Iodine (Potassium Iodide) | 150 μg |
| Magnesium (Magnesium Oxide) | 20 mg |
| Zinc (Zinc Oxide) | 25 mg |
| Copper (Copper Oxide) | 2.0 mg |
| Algal Oil Blend (from Crypthecodinium cohnii) | 415 mg* |

(*providing 200 mg DHA)

Soft Shell Capsule #1 may contain any one or more of the following optional ingredients: gelatin, soybean oil, sorbitol, glycerol, yellow beeswax, USP purified water, lecithin, titanium dioxide, FD&C Red #40, FD&C Blue #1, white edible ink, soy, corn oil, DL alpha tocopherol, and medium chain triglycerides.

Soft Shell Capsule #2

| | |
|---|---|
| Docusate sodium | 50 mg |

Soft Shell Capsule #2 may contain any one or more of the following optional ingredients: gelatin, sorbitol, polyethylene glycol, glycerin, purified water, propylene glycol, titanium dioxide, citric acid, and edible ink.

### Example 3

A composition of the following formulation was prepared in soft shell capsule form, including the appropriate excipients, by standard methods known to those of ordinary skill in the art:

A first composition of the following formulation is prepared in an appropriate oral dosage form is prepared, including the appropriate excipients, by standard methods known to those of ordinary skill in the art:

| | |
|---|---|
| Vitamin A (Beta Carotene) | 1100 IU |
| Vitamin C | 30 mg |
| Vitamin D (Vitamin D3) | 1000 IU |
| Vitamin E | 20 IU |
| Vitamin B1 | 1.6 mg |
| Vitamin B2 (Riboflavin) | 1.8 mg |
| Vitamin B3 (Niacinamide) | 15 mg |
| Vitamin B6 | 2.5 mg |
| Vitamin B9 (Folic Acid) | 1 mg |
| Vitamin B12 | 12 μg |
| Iron | 29 mg, 60 mg or 90 mg |
| Magnesium | 20 mg |
| Zinc | 25 mg |
| Copper | 2.0 mg |
| Iodine | 150 μg |
| DHA | 200 mg |

A second composition of the following formulation is prepared in an appropriate oral dosage form is prepared, including the appropriate excipients, by standard methods known to those of ordinary skill in the art:

| | |
|---|---|
| Docusate sodium | 15 mg, 30 mg or 50 mg |

### Example 4

In another example, various active ingredients may be incorporated into multiple compositions as a kit. In this non-limiting example, a first composition of the following formulation is prepared in caplet form, including the appropriate excipients, by standard methods known to those of ordinary skill in the art:

| | |
|---|---|
| Vitamin A (Beta Carotene) | 1100 IU |
| Vitamin C | 30 mg |
| Vitamin D (Vitamin D3) | 1000 IU |
| Vitamin E | 20 IU |
| Vitamin B1 | 1.6 mg |
| Vitamin B2 (Riboflavin) | 1.8 mg |
| Vitamin B3 (Niacinamide) | 15 mg |
| Vitamin B6 | 2.5 mg |
| Vitamin B9 (Folic Acid) | 1 mg |
| Vitamin B12 | 12 μg |
| Iron | 29 mg, 60 mg or 90 mg |
| Magnesium | 20 mg |
| Zinc | 25 mg |
| Copper | 2.0 mg |
| Iodine | 150 μg |

A second composition of the following formulation is prepared in soft shell capsule form by standard methods known to those of ordinary skill in the art:

| | |
|---|---|
| DHA | 200 mg |

A third composition of the following formulation is prepared in an appropriate oral dosage form is prepared, including the appropriate excipients, by standard methods known to those of ordinary skill in the art:
Docusate sodium 15 mg, 30 mg or 50 mg

### Example 5

In another example, various active ingredients may be divided into multiple compositions or a kit. In this non limiting example, the active ingredients of the composition of Example 3 may be divided into multiple compositions or kits. In this non limiting example, a first composition of the following formulation is prepared in soft shell capsule form, including the appropriate excipients, by standard methods known to those of ordinary skill in the art:

| | |
|---|---|
| Vitamin A (Beta Carotene) | 550 IU |
| Vitamin C | 15 mg |
| Vitamin D (Vitamin D3) | 500 IU |
| Vitamin E | 10 IU |
| Vitamin B1 | 0.8 mg |
| Vitamin B2 (Riboflavin) | 0.9 mg |
| Vitamin B3 (Niacinamide) | 7.5 mg |
| Vitamin B6 | 1.25 mg |
| Vitamin B9 (Folic Acid) | 0.5 mg |
| Vitamin B12 | 6.0 μg |
| Iron | 14.5 mg or 45 mg |
| Magnesium | 10 mg |
| Zinc | 12.5 mg |
| Copper | 1.0 mg |
| Iodine | 75 μg |
| DHA | 100 mg |

A second composition of the following formulation is prepared in soft shell capsule form by standard methods known to those of ordinary skill in the art:

| | |
|---|---|
| Vitamin A (Beta Carotene) | 550 IU |
| Vitamin C | 15 mg |
| Vitamin D (Vitamin D3) | 500 IU |
| Vitamin E | 10 IU |
| Vitamin B1 | 0.8 mg |
| Vitamin B2 (Riboflavin) | 0.9 mg |
| Vitamin B3 (Niacinamide) | 7.5 mg |
| Vitamin B6 | 1.25 mg |
| Vitamin B9 (Folic Acid) | 0.5 mg |
| Vitamin B12 | 6.0 μg |
| Iron | 14.5 mg or 45 mg |
| Magnesium | 10 mg |
| Zinc | 12.5 mg |
| Copper | 1.0 mg |
| Iodine | 75 μg |
| DHA | 100 mg |

A third composition of the following formulation is prepared in an appropriate oral dosage form is prepared, including the appropriate excipients, by standard methods known to those of ordinary skill in the art:

| | |
|---|---|
| Docusate sodium | 25 mg or 50 mg |

### Example 6

A study is undertaken to evaluate the effectiveness of the compositions of the present invention in the treatment of patients. The objective of the study is to determine whether oral intake of the compositions results in an improvement of the nutritional status of patients with regard to the specific vitamins and minerals contained in the administered compositions.

A double-blind, placebo controlled study is conducted over a six-month period. A total of 120 subjects (60 pregnant women entering the second trimester of pregnancy and 60 lactating women), aged 20-35 years, are chosen for the study. An initial assessment of the nutritional status of each woman is conducted. Vitamin A and vitamin B6 are measured using high performance liquid chromatography. Erythrocyte transketolase activity is used to measure vitamin B1 levels. Vitamin B2 levels are determined by assessment of erythrocyte glutathione reductase activity. Vitamin B3 levels are assessed by measuring urinary excretion of N′-methylnicotinamide and its pyridone. Vitamin B9 is measured by radioimmunoassay (RIA), specifically. The Solid Phase No Biol Folic Acid Kit (Diagnostic Products, Los Angeles, Calif.). Vitamin B12 is measured by RIA using human intrinsic factor as a binder and also by measuring serum levels of vitamin B12. Vitamin C levels are measured by spectrophotometric and colorimetric methods and also by measuring serum levels of Vitamin C. Vitamin D is measured using an extraction double-antibody RIA (Dia Sorin, Inc., Stillwater, Minn.). The peroxide hemolysis test is used to determine vitamin E status. Iron levels are measured using standard spectrophotometry. Iodine levels are measured by HPLC. Magnesium levels are measured by absorbance of a magnesium chelate with xylidl blue at 660 nM. Zinc levels are assessed using flame atomic absorption spectrometry (Perkins Elmer 460, Norwalk, Conn.). DHA is measured and quantified using gas chromatography procedures. Iron levels are measured by one or a combination of hemoglobin or hemocrit tests.

Additionally, total serum homocysteine levels are determined by extraction on the Multi-Prep® gravity series GVSA-100 column, a strong anion exchange gravity flow column, and measurement by gas chromatography/mass spectrometry. Biochemical Diagnostics, Austin, Tex.

The 120 subjects are separated into four separate groups of 30 women. In a first group comprising only pregnant women and in a second group comprising only lactating women, each subject is administered one dosage form of the composition of soft shell capsule #1 as described in Example 1 once a day. In a third group comprising only pregnant women and in a fourth group comprising only lactating women, each subject is administered one placebo dosage form once a day. Thus, dosage form administration occurs every 24 hours. No other nutritional supplements are taken by the subjects during the assessment period.

An assessment of the nutritional status of each woman is conducted utilizing the methods described above at one month intervals for a six month period. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1, 2, 3, 4, 5, and 6 months, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

An unexpected statistically significant improvement in the nutritional status of vitamin, mineral, and nutrient levels measured is observed in the treated subjects over the controls upon completion of the study. The iron levels in women receiving supplements as measured by a hemoglobin test or a hemocrit test demonstrate no iron deficiency. Women receiving supplements demonstrate a hemoglobin test result of at least about 12.0 to about 15.5 grams per deciliter. Alternatively or in addition, women receiving supplements demonstrate a hemocrit test result of at least about 34.5 to 44.5%.

The co-administration of docusate sodium mitigates constipation that can result from iron supplementation as evidenced by the frequency of normal bowel movements in women both before and after receiving supplements to remain substantially the same.

Additionally, homocysteine levels in women receiving supplements remain unelevated. Therefore, the study confirms that oral administration of the compositions of the present invention is effective in improving the nutritional status of patients. Other unexpected results relate to the observation that the length of gestation is increased by approximately six days in women receiving supplements, due to DHA intake, and their homocysteine levels are not elevated, due to folic acid intake, leading to a better prognosis regarding risk of neural tube defects in their infants.

### Example 7

A study is undertaken testing supplementation of iron, vitamin B12 and vitamin C. A study is conducted over a three-month period. A total of 120 subjects (pregnant women entering the second trimester of, aged 20-35 years, are chosen for the study) An initial assessment of the nutritional status of each woman for iron, vitamin B12 and vitamin C is conducted as provided in Example 6.

In the first group, each subject is administered one dosage form of the composition of soft shell capsule #1 as described in Example 1 once a day. In the second group, each subject is administered 1 one caplet daily that includes the following ingredients in the listed amounts (control):

| | |
|---|---|
| Vitamin A (Beta Carotene) | 1100 IU |
| Vitamin C | 30 mg |
| Vitamin D (Vitamin D3) | 1000 IU |
| Vitamin E | 20 IU |
| Vitamin B1 | 1.6 mg |
| Vitamin B2 (Riboflavin) | 1.8 mg |
| Vitamin B3 (Niacinamide) | 15 mg |
| Vitamin B6 | 2.5 mg |
| Vitamin B9 (Folic Acid) | 1 mg |
| Vitamin B12 | 12 μg |
| Iron | 29 mg |
| Iodine | 150 μg |
| Magnesium | 20 mg |
| Zinc | 25 mg |
| Copper | 2.0 mg |
| DHA | 200 mg |

An assessment of nutritional status for each subject is measured at one-month intervals for a six month period. Specifically, in regard to iron deficiency, vitamin C deficiency and vitamin B12 deficiency. The data is evaluated using multiple linear regression analysis and a standard students t-test. In each analysis the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 16, 12, and 8 weeks, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status is preferably observed in the treated subjects who were administered the composition of Example 1 over treated subjects who are administered the controls. Specifically, in regard to iron supplementation, the iron levels in women receiving supplements as measured by a hemoglobin test or a hemocrit test demonstrate no iron deficiency. Women receiving supplements demonstrate a hemoglobin test result of at least about 12.0 to about 15.5 grams per deciliter. Alternatively or in addition, women receiving supplements demonstrate a hemocrit test result of at least about 34.5 to 44.5%. The tested serum levels of vitamin B12 are preferably in the range of at least 200-900 picograms per milliliter (pg/mL), including all values between 200 and 900 pg/mL. The tested serum levels of vitamin C are preferably in the range of at least 0.6-2.0 milligrams per deciliter (mg/dL), including all values between 0.6 and 2.0 mg/dL. In regard to vitamin B12, vitamin C and iron content, serum levels show an increased levels as compared to the control.

### Example 8

A study is undertaken to ascertain whether the co-administration of soft shell capsule #1 and soft shell capsule #2 in a first group of subjects resulted in reduced variation from normal bowel movement habits as compared to a single administration of soft shell capsule #1 without co-administration with soft shell capsule #2 in a second group of subjects. A total of 120 subjects (pregnant women entering the second trimester of, aged 20-35 years, are chosen for the study). An initial assessment of each subject's bowel movement habits is made, obtaining information for each subject the frequency of bowel movements per day and perceived exertion during bowel movements on a scale of 1-10. A baseline value was established for each subject.

In the first group, each subject is co-administered soft shell capsules #1 and 2 as described in Example 1 once a day. In the second group, each subject is administered soft shell capsule #1 as described in Example 1 once a day, along with a soft shell capsule as a placebo, having the size and structure of soft shell capsule #2 as described in Example 1, but without the docusate sodium. An assessment of the subject's bowel movement habits is measured at one-month intervals for a six-month period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant maintenance or improvement in the bowel movement habit is preferably observed in the first group of subjects, receiving co-administration of soft shell capsules #1 and 2 of Example 1 over the second group of subjects, receiving only the administration of the soft shell capsule #1 of Example 1. Specifically, in regard to the frequency of bowel movements, subjects in the second group receiving only soft shell capsule #1 without co-administration with soft shell capsule #2, experienced significant disruption from their baseline bowel movement habits, with an average decreased frequency of at least about 33% and an average increased level of perceived exertion of at least about 40% over the six month period. In contrast, subjects in the second group receiving co-administration of soft shell capsules #1 and #2 experienced significantly less disruption from their baseline bowel movement habits, with an average decreased frequency of no more than about 15% and an average increased level of perceived exertion of no more than about 20%. These results demonstrate that soft shell capsule #2, comprising docusate sodium, is effective in mitigating constipation in subjects receiving iron supplementation, particularly high iron supplementation.

While specific embodiments of the present invention have been described, other and further modifications and changes may be made without departing from the spirit of the invention. All further and other modifications and changes are included that come within the scope of the invention as set forth in the claims. The disclosure of each publication cited above is expressly incorporated by reference in its entirety to the same extent as if each were incorporated by reference individually.

1. A kit comprising:
a first outer coating encapsulating a first filler composition comprising vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids, and one or more pharmaceutically-acceptable carriers; and
a second outer coating encapsulating a second filler composition comprising a laxative and one or more pharmaceutically-acceptable carriers.

2. The kit of claim 1, wherein the first filling composition comprises at least about 1100 IU vitamin A, at least about 60 mg vitamin C, at least about 1000 IU vitamin D, at least about 20 IU vitamin E, at least about 1.6 mg vitamin B1, at least about 1.8 mg vitamin B2, at least about 15 mg vitamin B3, at least about 1 mg vitamin B9, at least about 25 μg vitamin B12, at least about 90 mg iron, at least about 150 μg iodine, at least about 20 mg magnesium, at least about 25 mg zinc, at least about 2 mg copper and at least about 400 mg of the source of omega-3 fatty acids.

3. The kit of claim 2, wherein the vitamin A is in the form of beta carotene, the vitamin C is in the form of ascorbic acid, the vitamin D is in form of cholecalciferol, the vitamin E is in the form of dl-alpha tocopheryl acetate, the vitamin B1 is in the form of thiamin, the vitamin B2 is in the form of riboflavin, the vitamin B3 is in the form of niacinamide, the vitamin B6 is in the form of pyridoxine hydrochloride, the vitamin B9 is provided as a combination of folic acid and L-methylfolate calcium, the vitamin B12 is in the form of cyanocobalamin, the iron is in the form of polysaccharide iron complex, the iodine is in the form of potassium iodide, the magnesium is in the form of magnesium oxide, the zinc is in the form of zinc oxide, the copper is in the form of copper oxide, the source of omega-3 fatty acids is an algal oil comprising Crypthecodinium cohnii.

4. The kit of claim 1, wherein
the vitamin A is in the form selected from one or more of the group consisting of retinol acetate, retinol, retinol palmitate, retinoic acid, retinal, beta-cryptoxanthin, alpha-carotene, beta-carotene, gamma-carotene, and provitamin A carotenoids;
the vitamin C is in the form selected from one or more of the group consisting of ascorbic acid, asorbates, calcium ascorbate, sodium ascorbate, dehydroascorbic acid and salts, ascorbyl palmitate, ascorbyl phosphates and salts, ascorbyl sulfates and salts, acylated ascorbic acid derivatives, 6-bromo-6-deoxy-L-ascorbic acid, and ascorbate salts;
the vitamin D is in the form selected from one or more of the group consisting of vitamin D3, vitamin D2, previtamin D2, ergosterol, calcitriol, 7-dehydrocholesterol, Vitamin D1, vitamin D4, vitamin, 7-dehydrositosterol, Lumisterol, 25-hydroxyvitamin D, all steroids that exhibit the biological activity of calciol, 25-fluorocalciol, (3S)-3-amino-3-deoxycalciol, 11α-acetoxycalciol, calcidiol, ercalcitriol, calcitetrol, tacalciol, (5E)-isocalciol, Dihydroercalciol, (1S)-Hydroxycalciol, (24R)-Hydroxycalcidiol, Ercalcidiol, Ercalcitriol, Ertacalciol, (5E)-(10S)-10,19-Dihydroercalciol, (6Z)-Tacalciol, and (22E)-(24R)-Ethyl-22,23-didehydrocalciol;
the vitamin E is in the form selected from one or more of the group consisting of alpha, beta, gamma, and delta tocopherols in its natural or synthetic (dl) forms; alpha, beta, gamma, and delta tocotrienols in its natural or synthetic (dl) forms, dl-alpha tocopheryl derivatives such as dl-alpha tocopheryl esters, dl-alpha-tocopheryl acetate or succinate and d-alpha-tocopheryl acetate or dl-alpha tocopheryl phosphates;
the vitamin B1 is in the form selected from one or more of the group consisting of thiamine, thiamine mononitrate, thiamine monophosphate, thiamine diphosphate, thiamine triphosphate, acetiamine, allithiamine, prosultiamine and S-acyl derivatives of thiamine such as benfotiamine, fursultiamine and salts and esters thereof;
the vitamin B2 is in the form selected from one or more of the group consisting of flavin mononucleotide (FMN), flavin adenine dinucleotide (FAD), riboflavin (also known as 7,8-dimethyl-10-((2R,3R,4S)-2,3,4,5-tetrahydroxypentyl) benzo [g] pteridine-2,4 (3H,10H)-dione or lactoflavin) and riboflavin derivatives such as riboflavin-5′-monophosphate, riboflavin-5′-monobutyrate and riboflavin-5′-monopalmitate;
the vitamin B3 is in the form selected from one or more of the group consisting of niacin, and nicotinamide and salts and esters thereof;
the vitamin B6 is in the form selected from one or more of the group consisting of pyridoxine, 3-hydroxy-4,5-bis(hydroxymethyl)2-methylpyridine, 5′-deoxypyridoxal, 2-demethylpyridoxal(2-norpyridoxal), 2-propyl-2-norpyridoxal (2′-ethylpyridoxal), 6-methylpyridoxal, 2′-hydroxypyridoxal (2-hydroxymethyl-2-demethylpyridoxal or 2-hydroxymethyl-2-norpyridoxal), 4′-deoxypyridoxine 5′-phosphate, 5′-methylpyridoxal-5′-phosphate, pyridoxal N-oxide 5′-phosphate, Pyridoxal, Pyridoxamine, Pyridoxine-5′-phosphate (PNP), pyridoxal-5′-phosphate (PLP) and pyridoxamine-5′-phosphate (PMP), and their salts and chelates thereof;
the vitamin B9 is in the form selected from one or more of the group consisting of folic acid, folinic acid, folacin, metafolin, and/or one or more natural isomers of folate including (6S)-tetrahydrofolic acid or a polyglutamyl derivative thereof, (6S,R)-tetrahydrofolic acid or a polyglutamyl derivative thereof, 5-methyl-(6S)-tetrahydrofolic acid or a polyglutamyl derivative thereof, 5-methyl-(6S,R)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof and their salts and esters thereof;
the vitamin B12 is in the form selected from one or more of the group consisting of cobalamin, methylcobalamin, 5′-deoxyadenosylcobalamin, cyanocobalamin, hydroxycobalamin and mecobalamin;
the iron is in the form selected from one or more of the group consisting of elemental iron, in the form of a salt, chelated form, non-chelated form, chelated to an amino acid, carbonyl iron, ferrous gluconate, ferrous fumarate, polysaccharide iron complex, elemental polysaccharide iron, polysaccharide iron, ferrous (II)-bis-glycinate chelate, ferrous asparto glycinate, ferrous bisglycinate, ferrous bisglycinate hydrochloride, ferrous bisglycinate, elemental ferrous bisglycinate, ferrous sulfate, ferronyl (micronized), as Iron Aid, iron protein succinylate, carbonyl iron, Sumalate iron, Heme iron complex, as Ferrochel amino acid chelate, Heme iron polypeptide as Proferrin-bovine source, as heme iron polypeptide (bovine source) as sodium iron EDTA (Ferrazone), ferric ammonium citrate, elemental iron, and ferric pyrophosphate;
the iodine is in the form selected from one or more of the group consisting of iodide, elemental iodine, iodized salt, Lugol's iodine, sodium iodide, potassium iodide, potassium iodate, nascent iodine, and Nano-Colloidal Detoxified Iodine;
the magnesium is in the form selected from one or more of the group consisting of elemental magnesium, in the form of a salt, in a chelated form, in a non-chelated form, magnesium acetate, magnesium carbonate, magnesium gluconate, magnesium chloride, magnesium citrate, magnesium silicate, magnesium stearate, magnesium sulfate, magnesium oxide, and magnesium chelated to an amino acid;
the zinc is in the form selected from one or more of the group consisting of elemental zinc, in the form of a salt, in a chelated form, in a non-chelated form, zinc acetate, zinc gluconate, zinc picolinate, zinc sulfate and zinc oxide;
the copper is in the form selected from one or more of the group consisting of elemental copper, in the form of a salt, in a chelated form, in a non-chelated form, cupric oxide, copper sulfate, copper gluconate, copper citrate, cupric acetate, and alkaline copper carbonate; and
the source of omega-3 fatty acids is selected from one or more of the group consisting of: one or more of animal, fish, plants, algae or microorganism production.

5. The kit of claim 4, wherein the source of the omega-3 fatty acids is algal oil from one or more algae selected from the group consisting of: Schizochytrium sp, Crypthecodinium cohnii, Ulkenia sp. SAM2179, Schizochytrium linacinum strain SC-1

6. The kit of claim 1, wherein the laxative comprises docusate sodium.

7. The kit of claim 6, wherein the laxative comprises at least about 50 mg of the docusate sodium.

8. A kit comprising:
a first composition comprising vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids and one or more pharmaceutically-acceptable carriers; and
an adjuvant composition formulated to mitigate at least one undesired side effect associated with the administering of the first composition to the patient.

9. The kit of claim 8, further comprising one or both of a first outer coating surrounding the first composition and an adjuvant outer coating surrounding the adjuvant composition

10. The kit of claim 9, wherein the one or both of the first outer coating and the adjuvant outer coating are separately selected from the group consisting of: a hard shell, a soft shell, and a coating.

11. The kit of claim 10, wherein the soft shell is a gelatin shell.

12. The kit of claim 10, wherein the coating is selected from the group consisting of: a polymeric coating, an enteric coating and a sugar coating.

13. The kit of claim 8, wherein the first composition comprises at least about 90 mg of iron.

14. The claim of claim 13, wherein the iron is in the form of a polysaccharide iron complex.

15. The kit of claim 13, wherein the at least one undesired side effect is constipation.

16. The kit of claim 8, wherein the adjuvant composition comprises one or a combination of fiber and a laxative.

17. The kit of claim 16, wherein the laxative is a stool softener and wherein the stool softener is docusate sodium.

18. A nutritional composition comprising:
an outer coating encapsulating a filler composition comprising vitamin A, at least 60 mg of vitamin C, vitamin D, vitamin E, vitamin B1, vitamin B2, vitamin B3, vitamin B6, vitamin B9, vitamin B12, at least about 90 mg of iron, iodine, magnesium, zinc, copper, a source of omega-3 fatty acids, and one or more pharmaceutically-acceptable carriers;
wherein the outer coating is selected from the group consisting of: a hard shell, a soft shell and a coating.

19. The nutritional composition of claim 18, further comprising an adjuvant composition comprising one or a combination of a fiber and a laxative.

20. The nutritional composition of claim 19, wherein the adjuvant comprises a laxative and the laxative is docusate sodium.

21. A method comprising:
administering one or both of a first nutritional composition and an adjuvant composition to a patient in need thereof,
wherein the first nutritional composition comprises vitamin A, vitamin C, vitamin D, vitamin E, vitamin B1, vitamin B2, vitamin B3, vitamin B6, vitamin B9, vitamin B12, iron, iodine, magnesium, zinc, copper, a source of omega-3 fatty acids; and
wherein the adjuvant composition is formulated to mitigate at least one undesired side effect associated with the administering of the first nutritional composition to the patient.

22. The method of claim 21, wherein the patient in need thereof is suffering from one or more disease states associated with a nutritional deficiency.

23. The method of claim 21, wherein the patient is a female human, and said administering is performed before pregnancy, during pregnancy, after pregnancy, while breast-feeding, or a combination thereof.

24. The method of claim 21, wherein the first nutritional composition comprises at least 90 mg of iron.

25. The method of claim 21, wherein the adjuvant composition is a laxative.

26. The method of claim 25, wherein the laxative is docusate sodium.

27. The method of claim 21, wherein the administering is co-administering both of the first nutritional composition and the adjuvant composition to the patient.

28. A kit comprising:
a first outer coating encapsulating a first filler composition consisting of: at least about 1100 IU vitamin A, at least about 60 mg vitamin C, at least about 1000 IU vitamin D, at least about 20 IU vitamin E, at least about 1.6 mg vitamin B1, at least about 1.8 mg vitamin B2, at least about 15 mg vitamin B3, at least about 2.5 mg vitamin B6, at least about 1 mg vitamin B9, at least about 25 mcg vitamin B12, at least about 90 mg iron as polysaccharide iron complex, at least about 150 mcg iodine, at least about 20 mg magnesium, at least about 25 mg zinc, at least about 2 mg copper, a source of omega-3 fatty acids from an algal oil, and one or more pharmaceutically-acceptable carriers; and
a second outer coating encapsulating a second filler composition consisting of: a laxative and one or more pharmaceutically-acceptable carriers.

29. The kit of claim 28, wherein the laxative is docusate sodium in an amount of at least about 50 mg.
